(19) United States
(12) Patent Application Publication
(10) Pub. No.: US 2023/0357800 A1
(43) Pub. Date: Nov. 9, 2023

(54) INTEGRATION OF RENEWABLE CHEMICAL PRODUCTION INTO OIL, GAS, PETROLEUM, AND CHEMICAL PROCESSING AND INFRASTRUCTURE

(71) Applicant: LanzaTech, Inc., Skokie, IL (US)

(72) Inventors: Sean Dennis Simpson, Evanston, IL (US); Zarath Morgan Summers, Prospect Heights, IL (US)

(21) Appl. No.: 18/313,154

(22) Filed: May 5, 2023

Related U.S. Application Data
(60) Provisional application No. 63/339,397, filed on May 6, 2022.

(51) Int. Cl. C12P 7/06; C12M 1/00; C12M 1/34; C12P 7/62; C12N 1/20

(52) U.S. Cl. CPC C12P 7/065; C12M 43/04; C12M 41/34; C12M 47/10; C12P 7/62; C12N 1/20

(57) ABSTRACT

Improving overall carbon capture and improving overall production yield in chemical manufacturing facilities by integrating microbial fermentation into existing oil and gas infrastructure. Converting carbon sources that would otherwise be vented to the atmosphere or discarded as waste to one or more products. In certain aspects, also disclosed are to processes for producing desirable products, such as ethylene, from industrial waste streams.

### CROSS REFERENCE TO RELATED APPLICATIONS

This application claims the benefit of U.S. Provisional Patent Application No. 63/339,397, filed May 6, 2022, the entirety of which is incorporated herein by reference.

### FIELD

The present disclosure relates to improving carbon capture and improving overall production yield in chemical manufacturing facilities by integrating into existing oil and gas infrastructure microbial fermentation that converts carbon sources that would otherwise be vented to the atmosphere or discarded to one or more products. More specifically, the present disclosure relates to systems and methods for integrating gas fermentation into oil and gas production, transportation and refining, and/or chemical complexes to convert underutilized resources into useful products, such as ethylene, ethanol, and the like.

### BACKGROUND

The following discussion is provided to aid the reader in understanding the disclosure and is not admitted to describe or constitute prior art thereto.

Gas fermentation processes can be used to generate target materials from gas substrates or other input materials, particularly carbon-based materials. For example, particular biological systems can be used to perform gas fermentation.

Industrial processes can output gases that have significant amounts of carbon-based materials. Chemical processors and oil refiners generally view flaring and venting carbon rich sources to the atmosphere or otherwise discarding them as traditional standard operations. Currently, the primary alternative available to chemical processors and oil refiners is to engage in some form of carbon capture and sequestration (“CCS”).

CCS can include finding permanent underground storage such as depleted oil wells or sealed saline aquifers to permanently store the gaseous carbon. This may be cost prohibitive for chemical processors, oil refiners, or any other operator that produces underutilized carbon, as it requires them to locate a suitable location, construct a pipeline to that location, and then monitor the location indefinitely for leaks or other signs of failure.

Additionally, many domestic and international governmental entities are placing tighter restrictions on the total amount of carbon that a particular site, complex, or entity is allowed to release into the atmosphere. Such restrictions are pushing industrial, commercial, and agricultural operators alike to pursue and implement expensive efficiency upgrades to already well-developed technologies within their respective fields.

Although gas fermentation processes can be used for carbon capture and other applications, gas fermentation could be more widely implemented if cost barriers of constructing and maintaining the infrastructure necessary to transport either the underutilized carbon source to a gas fermentation processing or transporting the products generated by the gas fermentation processes out to a buyer could be reduced or eliminated, such as by integration with systems and processes that an operator has or will have at a facility already.

### SUMMARY

Described herein are systems and methods for incorporating gas fermentation into oil and gas production and refining or chemical, such as petrochemical, complexes to convert various feedstocks, waste gas, and other gas by-products into useful products, such as ethylene, ethanol, and the like.

In a first aspect, the present disclosure provides methods of integrating a gas fermentation process with a chemical or refining process, the method comprising:
(a) providing a gaseous carbon source from the chemical or refining process to the gas fermentation process; or
(b) providing the gas fermentation product from the gas fermentation process to the chemical or refining process; or
(c) both providing the gaseous carbon source from the chemical or refining process to the gas fermentation process and providing the gas fermentation product from the gas fermentation process to the chemical or refining process;
wherein the gas fermentation process utilizes a C1-fixing microorganism to ferment at least the gaseous carbon source from the chemical or refining process to produce the gas fermentation product; and
wherein the chemical or refining process generates a chemical or refining product.

In some embodiments, the gaseous carbon source is provided from the chemical or refining process to the gas fermentation process, the method further comprising adding the gas fermentation product to the chemical or refining process.

In some embodiments, the gas fermentation product is the same as the chemical or refining product.

Some embodiments may comprise performing the gas fermentation process in a gas fermentation unit that receives the gaseous carbon source.

In some embodiments, producing, by the gas fermentation process, the gas fermentation product comprises producing, by the gas fermentation process, a mixture comprising at least one of the gas fermentation product or a product-enriched off-gas. In some embodiments, providing the gas fermentation product from the gas fermentation process to the chemical or refining process comprises recovering a carbon product from the mixture and adding the carbon product to the chemical or refining product.

In some embodiments, the C1-fixing microorganism is an anaerobic bacterium. In some embodiments, the C1-fixing microorganism is an aerobic bacterium.

In some embodiments, the methods may further comprise a step of determining the mass of the carbon product.

In some embodiments, the methods may further comprise a step of determining the volume of the carbon product.

In another aspect, the present disclosure also provides gas fermentation systems for integration with a chemical or refining process, comprising: a first pipe configured to receive a gaseous carbon stream from a facility of the chemical or refining process; a gas fermentation unit coupled with the first pipe to receive the gaseous carbon stream, the gas fermentation unit comprising a culture of at least one C1-fixing microorganism capable of producing an output comprising a gas fermentation product from the gaseous carbon stream; and a second pipe coupled with the gas fermentation unit having a connection to a chemical plant and configured to contain the gas fermentation product; wherein the second pipe is adapted to receive the output from the gas fermentation unit and transport the output to the chemical plant, such that the gas fermentation product can be isolated, recovered, processed, or any combination thereof at the chemical plant.

In some embodiments, the oil refinery or production facility and the chemical plant are geographically adjacent to each other.

In some embodiments, the oil refinery or production facility and the chemical plant are part of the same operating complex.

In some embodiments, the oil refinery or production facility and the chemical plant are within 1 mile, within 2 miles, within 3 miles, within 4 miles, or within 5 miles of each other.

In some embodiments, the gaseous carbon stream is a waste gas, flare gas, natural gas, or captured flared gas.

In some embodiments, the gaseous carbon stream comprises CO, CO₂, methane, or any combination thereof, and, optionally, H₂.

In some embodiments, the oil refinery or production facility is selected from a topping refinery, a hydro-skimming refinery, a conversion refinery, a deep conversion refinery, or a coking unit.

In some embodiments, the gaseous carbon stream originates from a flexicoker, a pressure swing absorption unit, a flare stream, captured flared gases, or a pressure relief safety valve.

In some embodiments, the C1-fixing microorganism is aerobic or anaerobic.

In some embodiments, the C1-fixing microorganism is selected from a genus of Clostridium, Moorella, Carboxydothermus, Ruminococcus, Acetobacterium, Eubacterium, Butyribacterium, Oxobacter, Methanosarcina, Methanosarcina, Desulfotomaculum, and Cupriavidus.

In some embodiments, chemical plant is selected from an olefins plant, a poly-olefins plant, a polypropylene plant, a polyethylene plant, a polymer plant, a high-density polyethylene plant, an oligomers plant, a nitriles plant, an oxides plant, or a styrenics plant.

In some embodiments, the gas fermentation product is transported to a steam cracker within the chemical plant.

In some embodiments, the gas fermentation product is selected from an alcohol, an acid, a diacid, an alkene, a terpene, an isoprene, and alkyne.

In some embodiments, the gas fermentation product is selected from ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1propanol, 1hexanol, 1octanol, chorismate-derived products, 3hydroxybutyrate, 1,3butanediol, 2-hydroxyisobutyrate or 2-hydroxyisobutyric acid, isobutylene, adipic acid, 1,3hexanediol, 3-methyl-2-butanol, 2-buten-1-ol, isovalerate, isoamyl alcohol, and monoethylene glycol, or any combination thereof.

In some embodiments the system may further comprise a flow meter configured to determine a property or concentration of the gas fermentation product.

In another aspect, the present disclosure provides methods of integrating a gas fermentation unit with a chemical or refining processing unit, the method comprising: providing a gaseous carbon source from a section of the chemical or refining processing unit; fermenting a portion of the gaseous carbon source into a gas fermentation product with a gas fermentation unit, wherein the gas fermentation unit utilizes a C1-fixing microorganism to convert at least a portion of the gaseous carbon source into the gas fermentation product, resulting in a fermented mixture; recovering the gas fermentation product from the fermented mixture; and adding the gas fermentation product to a product of the chemical or refining processing unit.

In some embodiments, the gas fermentation product is the same as the product of the chemical or refining process.

In some embodiments, producing, by the gas fermentation process, the gas fermentation product comprises producing, by the gas fermentation process, a mixture comprising at least one of the gas fermentation product or a product-enriched off-gas.

In some embodiments, providing the gas fermentation product from the gas fermentation process to the chemical or refining process comprises recovering a carbon product from the mixture and adding the carbon product to the chemical or refining product.

In some embodiments, the C1-fixing microorganism is an anaerobic bacterium.

In some embodiments, the C1-fixing microorganism is an aerobic bacterium.

In some embodiments the methods may further comprise a step of determining the mass of the gas fermentation product.

In some embodiments the methods may further comprise a step of determining the volume of the gas fermentation product.

In another aspect, the present disclosure provides gas fermentation systems for integration with a chemical or refining process, comprising: a first connection configured to receive a carbon source from the chemical or refining process; a gas fermentation unit configured to receive a gaseous carbon stream of the carbon source and utilize a C1-fixing microorganism to convert the gaseous carbon stream into a gas fermentation product; and a second connection configured to add the gas fermentation product to a product of the chemical or refining process.

In some embodiments, the carbon source comprises at least one of a solid or a liquid, and the gas fermentation system comprises a gasifier configured to generate the gaseous carbon stream from the carbon source.

In some embodiments, the gas fermentation product is the same as the product of the chemical or refining process.

In some embodiments, the gas fermentation unit is configured to generate the gas fermentation product to comprise a product-enriched off-gas.

In some embodiments, the gas fermentation unit is configured to generate a mixture comprising the gas fermentation product, the gas fermentation system further comprising an separator configured to recover the gas fermentation product from the mixture to add the gas fermentation product to the product of the chemical or refining process.

In some embodiments, the C1-fixing microorganism is an anaerobic bacterium.

In some embodiments, the C1-fixing microorganism is an aerobic bacterium.

In some embodiments the systems may further comprise a sensor configured to determine a mass of the gas fermentation product.

In some embodiments the systems may further comprise a sensor configured to determine a volume of the gas fermentation product.

The foregoing general description and following detailed description are exemplary and explanatory and are intended to provide further explanation of the disclosure as claimed. Other objects, advantages, and features will be apparent to those skilled in the art from the following brief description of the drawings and detailed description of the disclosure.

### BRIEF DESCRIPTION OF THE DRAWINGS

The following detailed description is merely exemplary in nature and is not intended to limit the various embodiments or the application and uses thereof. Furthermore, there is no intention to be bound by any theory presented in the preceding background or the following detailed description. The Figures have been simplified by the deletion of a large number of apparatuses customarily employed in a process of this nature which are not specifically required to illustrate the performance of the invention. Furthermore, the illustration of the process of this disclosure in the embodiment of a specific drawing is not intended to limit the invention to specific embodiments. Some embodiments may be described by reference to the process configuration shown in the figures, which relate to both apparatus and methods to carry out the disclosure. Any reference to method includes reference to an apparatus unit or equipment that is suitable to carry out the step, and vice versa.

FIG. 1 is an overview of the piping and associated components of an embodiment of a gas fermentation process integrated within a chemical manufacturing or oil refining complex.

FIG. 2 is an overview of the piping and associated components of an embodiment of a gas fermentation process integrated within a chemical manufacturing or oil refining complex with a catalytic process.

FIG. 3 is an overview of the piping and associated components of an embodiment of the gas fermentation process.

FIG. 4 is a flowchart of an embodiment of a method of using a gas fermentation process integrated within a chemical manufacturing or oil refining complex.

FIG. 5 is a flowchart of an embodiment of a method of using a gas fermentation process integrated within a chemical manufacturing or oil refining complex.

### DETAILED DESCRIPTION

The present disclosure provides systems and methods for improving carbon capture and improving overall production yield in refining and chemical manufacturing facilities by integrating into existing oil and gas infrastructure microbial fermentation that converts carbon sources that would otherwise be vented to the atmosphere or discarded to one or more products. More specifically, the present disclosure relates to systems and methods for incorporating gas fermentation into oil and gas production and refining and chemical complexes to convert various feedstock, waste gas, and other gas by-products into useful products, such as ethylene, ethanol, and the like.

### A. DEFINITIONS

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

The term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. In addition, the term “acetate” includes both acetate salt alone and a mixture of molecular or free acetic acid and acetate salt, such as the mixture of acetate salt and free acetic acid present in a fermentation broth as described herein.

The term “carbon capture” as used herein refers to the fixation and utilization of carbon including carbon from CO₂, CO, and/or CH₄ from a stream comprising CO₂, CO, and/or CH₄ and converting the CO₂, CO, and/or CH₄ into useful products.

The term “substrate comprising carbon monoxide” and like terms should be understood to include any substrate in which carbon monoxide is available to one or more strains of bacteria for growth and/or fermentation, for example.

The term “gaseous substrates comprising carbon monoxide” includes any gas which contains carbon monoxide. The gaseous substrate will typically contain a significant proportion of CO, preferably at least about 5% to about 100% CO by volume.

The term “C1 carbon” and like terms should be understood to refer to carbon sources that are suitable for use by a microorganism, particularly those of the gas fermentation process disclosed herein. C1 carbon may include, but should not be limited to, carbon monoxide (CO), carbon dioxide (CO₂), and methane (CH₄), methanol (CH₃OH), and formate (HCOOH).

The term “substrate comprising carbon dioxide” and like terms should be understood to include any substrate in which carbon dioxide is available to one or more strains of bacteria for growth and/or fermentation, for example.

The term “gaseous substrates comprising carbon dioxide” includes any gas which contains carbon dioxide. The gaseous substrate will typically contain a significant proportion of CO₂, preferably at least about 5% to about 100% CO₂ by volume.

The term “bioreactor” includes a fermentation device consisting of one or more vessels and/or towers or piping arrangements, which includes the continuous stirred tank reactor (CSTR), immobilized cell reactor (ICR), trickle bed reactor (TBR), bubble column, gas lift fermenter, membrane reactor such as hollow fiber membrane bioreactor (HFMBR), static mixer, or other vessel or other device suitable for gas-liquid contact.

The term “co-substrate” refers to a substance that, while not necessarily being the primary energy and material source for product synthesis, can be utilized for product synthesis when added to another substrate, such as the primary substrate.

The term “directly”, as used in relation to the passing of industrial off or gases to a bioreactor, is used to mean that no or minimal processing or treatment steps, such as cooling and particulate removal are performed on the gases prior to them entering the bioreactor (note: an oxygen removal step may be required for anaerobic fermentation).

The terms “fermenting,” “fermentation process,” “fermentation reaction,” and like terms as used herein, are intended to encompass both the growth phase and product biosynthesis phase of the process. As is described further herein, in some embodiments the bioreactor may comprise a primary bioreactor and a secondary bioreactor.

The term “nutrient medium” as used herein should be understood as the solution added to the fermentation broth containing nutrients and other components appropriate for the growth of the microorganism culture.

The terms “primary bioreactor” or “first reactor” as used herein this term is intended to encompass one or more reactors that may be connected in series or parallel with a secondary bioreactor. The primary bioreactors generally use anaerobic or aerobic fermentation to produce a product (e.g., ethylene, ethanol, acetate, etc.) from a gaseous substrate.

The terms “secondary bioreactor” or “second reactor” as used herein are intended to encompass any number of further bioreactors that may be connected in series or in parallel with the primary bioreactors. Any one or more of these further bioreactors may also be connected to a further separator.

The term “stream” is used to refer to a flow of material into, through and away from one or more stages of a process, for example, the material that is fed to a bioreactor. The composition of the stream may vary as it passes through particular stages. For example, as a stream passes through the bioreactor.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

### B. DISCLOSED SYSTEMS AND METHODS

Systems and methods in accordance with the present disclosure can be used to integrate gas fermentation systems with infrastructure that generates carbon-based materials, such as existing oil and gas processing infrastructure, refining infrastructure, petrochemical infrastructure, and infrastructure in complexes that combine any of the foregoing as well as plants, mills, refineries, or other infrastructure that generates carbon-based materials as outputs, including as waste. Integrating a gas fermentation (hereinafter referred to as “GF”) unit with existing infrastructure allows users and operators to reduce their overall carbon emissions by converting their waste carbon into marketable products without additional capital investment and operating costs for equipment and processes that can be leveraged from infrastructure an operator may already have or is building for another primary purpose. In addition, any off gas produced downstream of a GF unit would be potentially better suited for traditional carbon capture units as it would be free of gas contaminants (H₂S) and have any CO removed. For some operators, integration can also increase their overall production yield if the product produced by the GF process is a product that the operator is capable of producing or is a feed component to a process an operator is already performing.

For many industrial processes, emission of gases that are rich in carbon are commonplace. Chemical processors and oil refiners generally view flaring and venting carbon rich sources to the atmosphere or otherwise discarding them as traditional standard techniques. Currently, the primary alternative available to chemical processors and oil refiners is to engage in some form of carbon capture and sequestration (“CCS”).

CCS involves finding permanent underground storage such as empty oil wells, gas-tight saline aquifers, or salt domes to permanently store the waste gaseous carbon. This option is generally cost prohibitive for chemical processors, oil refiners, or any other operator that produces waste carbon, as it requires them to locate a suitable location, construct a pipeline to that location, and then monitor the location indefinitely for leaks or other signs of failure.

Additionally, many domestic and international governmental entities are placing tighter restrictions on the total amount of waste carbon that a particular site, complex, or entity is allowed to release into the atmosphere. Such restrictions are pushing industrial, commercial, and agricultural operators alike to pursue and implement expensive efficiency upgrades to already well-developed technologies within their respective fields.

Gas fermentation processes that are capable of converting various carbon sources into other products are rapidly becoming a desirable alternative for producers of excess carbon. Such processes allow companies to convert standard techniques that emit carbon into the atmosphere into a separate revenue stream by converting the underutilized carbon into a marketable product. Moreover, the carbon that is converted into other products lowers the operator's total carbon output, potentially serving as a way for operators to maintain current outputs without conflicting with ever-tightening government regulations. Furthermore, tail gas from gas fermentation may be treated to form a concentrated CO₂ stream depleted in CO and/or sulfur thereby reducing the cost of more traditional carbon capture and sequestration processes.

The widespread adoption of gas fermentation processes could be improved by reducing the cost barriers of constructing and maintaining the infrastructure necessary to transport either the carbon source to a gas fermentation processing plant, or transporting the products generated by the gas fermentation processes out to a buyer.

In one embodiment the substrate and/or C1 carbon source is obtained from the refining or chemical operations in which the gas fermentation process is being integrated. In other embodiments, the substrate and/or C1 carbon source is obtained from outside the refining or petrochemical operations in which the gas fermentation process is being integrated and it is the product of the gas fermentation process that is being integrated with infrastructure of the refining or chemical operations. In yet other embodiments it is both the substrate and/or C1 carbon source. The substrate and or C1 carbon source may be already in the form of a gas (e.g., a waste gas or underutilized gas), or a solid or liquid material may be first processed in a preliminary step of the overall gas fermentation process to generate synthesis gas known as syngas which in turn is provided to the bioreactor of the gas fermentation system. The preliminary step to generate syngas may involve, reforming, partial oxidation, plasma, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material, gasification of tires, pieces of tires, and or components of tires, and gasification of tires, pieces of tires, and or components of tires combined with an organic material. Examples of reforming processes include, steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibers such as in shoes, apparel, and textiles. Municipal solid waste may be simply landfill-type waste and may be sorted or unsorted. Examples of tires include end of life tires, defective tires, surplus tires, and tire scraps. Examples of biomass may include lignocellulosic material and microbial biomass. Lignocellulosic material may include agriculture waste and forest waste.

The substrate and/or C1-carbon source may be a gas stream comprising methane. Such a methane containing gas may be obtained from fossil methane emissions such as during fracking or other hydrocarbon well stimulation processes or from coalbeds, or may be obtained from wastewater treatment, livestock, agriculture, and municipal solid waste landfills. It is also envisioned that the methane may be burned or employed as a feed in a fuel cell to produce electricity or heat, and the C1 by-products may be used as the substrate or carbon source.

The method of the invention may be used to produce one or more products. For instance, the products may include ethanol, acetate, 1-butanol, 2,3-butanediol, lactate, butene, butadiene, methyl ethyl ketone (2-butanone), ethylene, acetone, isopropanol, lipids, 3-hydroxypropionate (3-HP), terpenes, including isoprene, fatty acids, 2-butanol, 1,2-propanediol, 1 propanol, 1 hexanol, 1 octanol, chorismate-derived products, 3 hydroxybutyrate, 1,3 butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, and/or monoethylene glycol. In certain embodiments, microbial biomass itself may be considered a product. The microorganism of the disclosure may be cultured with the gaseous substrate to produce one or more carbon products. For instance, the microorganism of the disclosure may produce or may be engineered to produce ethanol, acetate, 1-butanol, 2,3-butanediol, lactate, butene, butadiene, methyl ethyl ketone (2-butanone), ethylene, acetone, isopropanol, lipids, 3-hydroxypropionate (3-HP), terpenes, including isoprene, fatty acids, 2-butanol, 1,2-propanediol, 1 propanol, 1 hexanol, 1 octanol, chorismate-derived products, 3 hydroxybutyrate, 1,3 butanediol, 2-hydroxyisobutyrate or 2-hydroxyisobutyric acid, isobutylene, adipic acid, 1,3 hexanediol, 3-methyl-2-butanol, 2-buten-1-ol, isovalerate, isoamyl alcohol, and/or monoethylene glycol. In certain embodiments, microbial biomass itself may be considered a product.

### C. SYSTEMS AND METHODS OF A GF UNIT INTEGRATED WITHIN A CHEMICAL MANUFACTURING OR OIL-REFINING COMPLEX

In certain embodiments, industrial process have feedstocks or gas (e.g., waste gas) suitable as substrate and/or C1 carbon source and are selected from ferrous metal products manufacturing, such as a steel manufacturing, non-ferrous products manufacturing, petroleum production, petroleum refining, electric power production, carbon black production, paper and pulp manufacturing, ammonia production, methanol production, coke manufacturing, coke gasification, petrochemical production, polymer production, ethylene production, olefin production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, landfill operations, or any combination thereof.

Examples of specific processing steps within an industrial process which may generate substrate and/or C1 carbon source for gas fermentation include catalyst regeneration and fluid catalyst cracking. Air separation and direct air capture are other suitable industrial processes to provide substrate and/or C1 carbon source for the gas fermentation process. Specific examples in steel and ferroalloy manufacturing include blast furnace gas, basic oxygen furnace gas, coke oven gas, direct reduction of iron furnace top-gas, and residual gas from smelting iron. In petroleum and oil production, C1 carbon may be produced with the oil or may be produced from a well separately. Other general examples include flue gas from fired boilers and fired heaters, such as natural gas, oil, or coal fired boilers or heaters, and gas turbine exhaust. In these embodiments, the substrate and/or C1-carbon source may be captured from the industrial process before it is emitted into the atmosphere, using any known method.

It is also envisioned that the product of the gas fermentation process may provide the point of integration. Products listed above as capable of being produced by gas fermentation may be reactants or components used in refining or chemical processes and integrating the product into a refining or chemical process may take advantage of equipment and operations in common with both the gas fermentation and the refining or chemical process.

Points of integration, as either providing the GF substrate and/or carbon source or as accepting the product of GF or both include the following. The integration may involve the GF substrate and/or carbon source from one refining or chemical process and the acceptance of the GF product into a second different refining or chemical process. Exemplary processes include distillation, including as examples, at atmospheric pressure, at reduced pressure, stripping, rerunning, stabilization, super fractionation, azeotropic, extractive, or any combination thereof; thermal cracking including for example, Visabreaking, coking, delayed coking, fluid coking, coke gasification, Flexicoking™ process, Aquaconversion™ process, Asphalt Coking Technology Process (ASCOT) Process, Cherry-P Process, Continuous Coking Process, Decarbonizing Process, ET-II Process, Eureka Process, FTC Process, HSC Process, Mixed-Phase Cracking Process, Shell Thermal Cracking Process, Tervahl-T Process, or any combination thereof; catalytic cracking as exemplified by fixed bed, moving bed, fluid bed, coke formation, Asphalt Residual Treating (ART) Process, Residue Fluid Catalyst Cracking Process, Heavy Oil Treating Process, R2R Process, Reduced Crude Oil Conversion Process, Shell FCC Process, and S&W Fluid Catalytic Cracking Process, or any combination thereof; hydrotreating as exemplified by hydrodesulfurization in downflow fixed-bed, upflow expanded-bed, distillate hydrodesulfurization, heavy feedstock hydrodesulfurization, Resid Desulfurization Process, Vacuum Resid Desulfurization Process, Residfining Process; biodesulfurization, or any combination thereof; hydrocracking as exemplified by Aquaconversion Process, Asphaltenic Bottom Cracking Process, CANMET Process, Chevron RDS Isomax Process, Chevron VRDS Process, ENI Slurry-Phase Technology, Gulf Resid Hydrodesulfurization Process, H-G Hdrocracking Process, H-Oil Process, HYCAR Process, Hyvahl-F Process, IFP Hydrocracking Process, Isocracking Process, LC-Fining Process, MAKfining Process, Microcat-RC Process, Mild Hydrocracking Process, MRH Process, RCD Unibon Process, Residfining Process, Residue Hydroconversion Process, Shell Residual Oil Process, Tervahl-H Process, Unicracking Process, Uniflex Process, Veba Combi Cracking Process, or any combination thereof; solvent processes as exemplified by desphalting processes, Deep Solvent Deasphalting Process, Demex Process, MDS Process, Residuum Process, Solvahl Process, Lube Deasphalting, dewaxing processes, Cold Press Process, Solvent Dewaxing Process, Urea Dewaxing Process, Centrifuge Dewaxing Process, Catalytic Dewaxing Process, dewaxing heavy feedstocks, or any combination thereof; product improvement processes including reforming, isomerization, hydroisomerization, alkylation, polymerization, treating, acid processes, clay processes, oxidative processes solvent processes, or any combination thereof; gasification processes including primary gasification, secondary gasification, water-gas shift reactions, carbon dioxide gasification, hydrogasification, methanation, Fischer-Tropsch synthesis, gasification of residua and residua, gasification of residua with biomass, gasification of residual with waste, or any combination thereof; hydrogen production and hydrogen purification; electricity, heat, or other energy production via combustion, gasification, or fuel cells; gas cleaning processes as exemplified by enrichment, water removal including, for example, adsorption and membrane, liquid removal including, for example, extraction, absorption and fractionation, nitrogen removal, acid gas removal, fractionation, and Claus Process.

Examples of reforming processes include thermal reforming, catalytic reforming in fixed bed mode and in moving bed mode, and fluid bed reforming. Examples of isomerization processes include Butamer Process, Butomerate Process, Hysomar Process, Iso-Kel Process, Isomate Process, Isomerate Process, Penex Process, and Pentafining Process. Examples of alkylation processes include sulfuric acid alkylation and hydrofluoric alkylation. Examples of polymerization processes include thermal, solidphosporic acid, and bulk acid. Examples of caustic treating processes include Dualayer Distillate Process, Dualayer Gasoline Process, Electrolytic Mercaptan Process, Ferrocyanide Process, Lye Treatment, Mercapsol Process, Polysulfide Process, Sodasol Process, Solutizer Process, Steam-Regenerative Caustic Treatment, and Unisol Process. Examples of acid treating processes include Nalfining Process, and Sulfuric Acid Treatment. Example of Clay treating processes include Alkylation Effluent Treatment, Arosorb Process, bauxite treatment, continuous contact filtration process, cyclic adsorption process, gray clay treatment, percolation filtration process, and Thermofor Continuous Percolation Process. Examples of oxidative treatment processed include Bender Process, Copper Sweetening Process, Doctor Process, Hypochlorine Sweetening Process, Inhibitor Sweetening Process, and Merox Process.

Refining processes listed are known and described in Speight, James G. Handbook of Petroleum Refining. Taylor & Francis, 2017.

Gas fermentation may be integrated with the following chemical manufacturing exemplary processes: alkylation, amination, condensation and addition, dehydration, dehydrogenation, esterification, ethynylation, other fermentation processes, Friedel-Crafts reactions, halogenation, hydration, hydrolysis, hydroformylation, hydrogenation, nitration, oxidation, oxo reaction, polymerization, sulfonation, and vinylation. Such chemical processes are known and described in Speight, James G. Chemical Process and Design Handbook, The McGraw-Hill Companies Inc, 2002.

Products made or resulting from any of the above chemical processes may include, but are not limited to, a commodity chemical selected from ethanol, isopropanol, monoethylene glycol, sulfuric acid, propylene, sodium hydroxide, sodium carbonate, ammonia, benzene, acetic acid, ethylene, ethylene oxide, formaldehyde, methanol, or any combination thereof. In another embodiment, the commodity chemical is incorporated into one or more articles, converted into one or more second products, or any combination thereof. In one embodiment, the one or more articles, the one or more second products, or any combination thereof, are selected from acetate, 1-butanol, butyrate, 2,3-butanediol, lactate, butene, butadiene, methyl ethyl ketone, acetone, lipids, 3-hydroxyproprionate, terpenes, isoprene, fatty acids, 2-butanol, 1,2-propanediol, 1 propanol, 1 hexanol, 1 octanol, chorismite-derived products, 3-hydroxybutyrate, 1,3-butanediol, 2-hydroxyisobutyrate, 2-hydroxyisobutyric acid, isobutylene, adipic acid, keto-adipic acid, 1,3-hexanediol, 3-methyl-2-butanol, 2-buten-1-ol, isovalerate, isoamyl alcohol, or any combination thereof.

Gas fermentation may be used to produce desirable products on site or off site. As a non-limiting example, in one embodiment, gas fermentation may be employed to convert methane directly from a gas or oil well into valuable products with possible carbon dioxide as a by-product. In such an embodiment, the gas fermentation reactor may be located at the well site, or may be remote to the well site. The carbon dioxide itself may be converted into products by gas fermentation, or released to the atmosphere, or may be advantageously used as part of further processes at the well site, such as for enhanced oil recovery operations.

In another example, in one embodiment, gasification of tires, pieces of tires, and or components of tires, optionally in combination with organic material, followed by gas fermentation may be employed to convert end of life tires, defective tires, and or tire scraps into valuable products. The gasification and gas fermentation process may be co-located with and integrated with chemical production processes used to generate chemicals and intermediates for use in the generation of new tires. Processing units in common with the gasification and gas fermentation process and the chemical production process may be integrated with each of the processes so that fewer units in total are needed. Capital equipment costs and operating costs may be minimized.

In yet another example, the gas fermentation system may be co-located with an energy production facility, such as a power plant, that generates carbon dioxide as an undesired by-product. The carbon dioxide may be provided to the gas fermentation process as feedstock/substrate along with hydrogen. The hydrogen may be from any source such as green hydrogen (from solar, wind or water), grey hydrogen (from natural gas or methane), blue hydrogen (from natural gas or methane with carbon capture), brown hydrogen (gasification), black hydrogen (from coal), and or turquoise hydrogen (from methane pyrolysis). The gas fermentation system is located at the source of the carbon-dioxide substrate eliminating need for transport of carbon dioxide. Additionally, end of life tires may be collected or transported to the site for gasification and production of additional feedstock or substrate to the gas fermentation process. The tire industry is in need of reducing carbon dioxide footprint, and in capturing and transforming the carbon dioxide produced in suppling the energy requirement to the tire manufacturing industry while at the same time recycling end of life tires into valuable chemicals that may be used to produce new tires, the tire industry may reduce its overall carbon emissions.

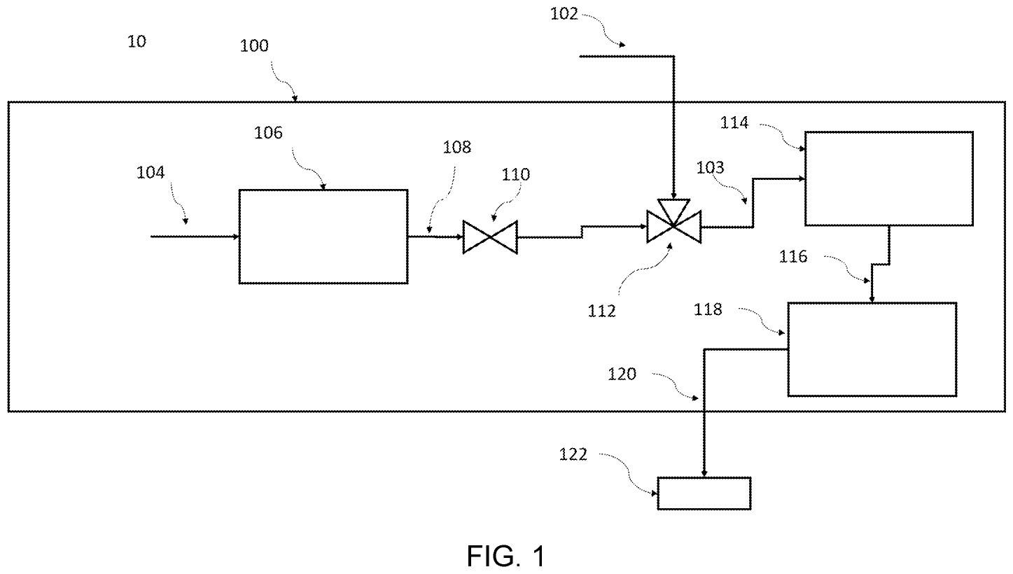

Referring now to FIG. 1, which illustrates an integration of a GF system 10 within a chemical manufacturing or oil refining complex 100. The system includes a gas stream 104 that is collected, captured, or harvested from a vessel or by-product process within the complex 100. The gas stream 104 is directed to the GF unit 106 as an input or feed stream. The GF unit 106 converts a portion of gas stream 104 into an output mixture 108 comprising of at least the product that the GF unit 106 is adapted to produce. To operate, the GF unit 106 may require other utility inputs such as compressed air, instrument air, an electrical feed, or other utility inputs commonly known by those skilled in the art. The output mixture may then be passed (e.g., pumped) to an optional metering valve 110 to determine the amount of product that is contained in the output mixture 108. The output mixture 108 then passes to mixing valve 112 where it mixes with incoming feedstock 102 for the chemical manufacturing or oil refining complex 100. The mixed feedstock 103 comprising the feedstock 102 and GF unit output mixture 108 is then passed (e.g., pumped) through a chemical or refinery processing unit 114 such that the mixed feedstock can be converted into a product and the product contained in the GF unit output mixture 108 can be recovered. The combined product 116 from both the feedstock 102 and the GF unit output mixture 108 is then passed (e.g., pumped) to an optional storage tank 118 prior to final sale and transport to the customer 122. In this way, the product of the GF process is handled along with the product of the chemical or oil refining process, using the same equipment already required for the chemical or oil refining process. Dedicated infrastructure for gas fermentation alone is minimized.

The optional storage tank 118 can be underground saline aquifers, spherical pressure containing vessels, cylindrical pressure vessels, rail cars, available portion of pipeline, barge, or any other suitable form of short, medium, or long term storage either on-site or off-site.

Metering valve 110 can include a ball valve, a check valve, a needle valve, plug valve, gate valve, or any other suitable type of valve to control the flow rate of the output mixture 108 of the GF unit 106. Metering valve 110 can either be manual control requiring input from a physical operator to function or it can be computer controlled such that the distributed control system (“DCS”) controls the function of the valve. Metering valve 110 can also include a Coriolis type flow meter, an electromagnetic type flow meter, an ultrasonic type flow meter, fluid velocity flow meter, inferential pressure type flow meter, pressure differential type flow meters, positive displacement type flow meter, or any other suitable flow meter type.

Varying the feed streams 104 to each GF unit 106 can be done to increase the feed 104 to GF units 106 that are adapted to produce products that readily meet or exceed certain target thresholds. Conversely, the feed 104 to GF units 106 configured to convert the feed stream 104 into products that do not meet certain target thresholds can be throttled down or halted completely. The various output mixtures 108 from the various GF units 106 can be configured to be passed to individual storage tanks (not shown) for short, medium, or long term storage, pumped directly into a pipeline, barge port, or other industrial infrastructure capable of transporting hydrocarbons.

The GF unit 106 can be configured to produce fuels that can be utilized on-site by the chemical manufacturing or oil refining complex 100. Such synergy allows for the complex 100 to lower the overall operating costs by decreasing the spend related to such fuels and fuel sources, thereby increasing the overall profitability of the complex 100.

A GF unit 106 can receive a feed 104 from a pressure relief safety valve (“PRSV”). A PRSV is typically installed on a pressure containing vessel and is adapted to open at a set pressure value to protect the vessel it is connected to. Typically, the streams contained within the pressure vessel are carbon rich and are routed to a safe relief point, either a flare source or the atmosphere. Operators can further increase their site productivity and efficiency by harvesting these otherwise wasted streams of carbon rich material by routing them to the integrated GF unit 106 to be converted into marketable products.

Multiple GF units 106 can be integrated within a single chemical manufacturing or oil refining complex 100. The multiple GF units 106 can be adapted to produce a similar or identical product from various different feed streams 104. The multiple GF units 106 can then have their respective outputs 108 configured to output to a single storage tank or processing unit 114. This synergy can allow the production operator to capture a maximum amount of waste gases or streams and convert it into a marketable product.

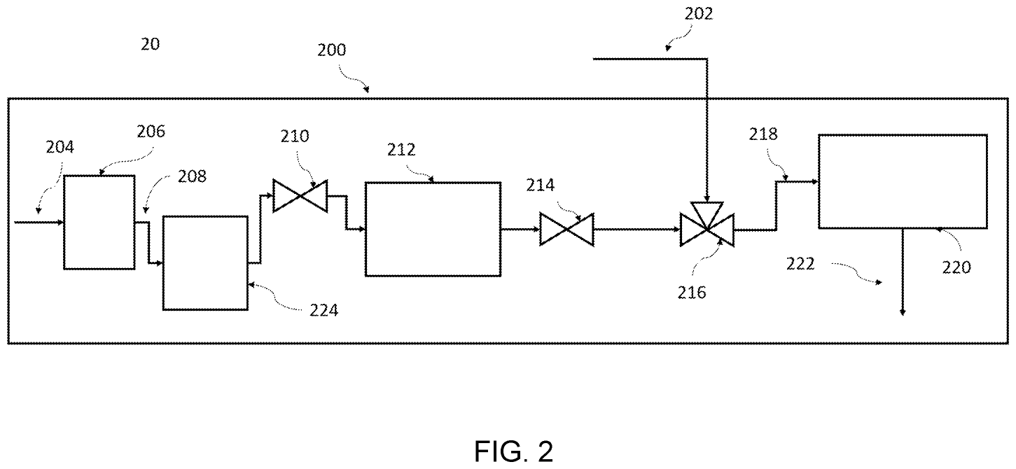

Referring now to FIG. 2, which illustrates an integration of a GF system 50 within a chemical manufacturing or oil refining complex 200 connected to a pipeline 202 configured to transport oil, natural gas, or other gaseous or liquid hydrocarbons from a production site to a processing unit 220. The system includes a carbon source 204 that is collected, captured, or harvested from a vessel or by-product process within the complex 200. The carbon source 204 is passed to GF unit 206 as an input or feed stream. The GF unit 206 converts a portion of carbon source 204 into an output mixture 208 that includes the product that the GF unit 206 is adapted to produce. The output mixture 208 is then passed to a metering valve 210 to determine the amount of product that is contained in the output mixture 208. The output mixture 208 is then passed to a catalytic process unit 224, which facilitates a reaction with a catalyst and the output mixture 208 into product molecules such as propane or BTEX. The output mixture 208 is then passed (e.g., pumped) to storage tank 212 where it can be stored for short, medium, or long term storage until the output mixture meets or exceeds target thresholds. Once the contents of the storage tank 212 meet or exceed target thresholds, the output mixture 208 is passed (e.g., pumped) through metering valve 214 to determine the amount of product that is being passed (e.g., pumped) from the storage tank 212. The output mixture 208, is then passed (e.g., pumped) to mixing valve 216 where output mixture 208 mixes with the contents of pipeline 202. The mixed contents 218 are then passed (e.g., pumped) to the processing unit 220 such that the contents of pipeline 202 can be converted into a product and the product contained in the GF unit output mixture 208 can be recovered as overall product stream 222. In this way, the product of the GF process is handled along with the product of the chemical process, using the same equipment already required for the chemical process. Dedicated infrastructure for gas fermentation alone is minimized.

The catalytic process unit 224 can be a device consisting of one or more vessels and/or towers or piping arrangements, which includes the continuous stirred tank reactor (CSTR), immobilized cell reactor (ICR), trickle bed reactor (TBR), bubble column, gas lift fermenter, membrane reactor such as hollow fiber membrane bioreactor (HFMBR), static mixer, or other vessel or other device suitable for gas-liquid contact. Fixed bed, moving bed, simulated moving bed, fluidized bed, entrained bed, slurry reactor, packed bed, trickle bed, batch, semi batch, continuous, plug flow, flash, dense phase, fixed bed, downflow fixed bed, upflow expanded bed, and ebullating bed.

The types of catalysts used in the catalytic process unit 224 can include, but are not limited to, natural clays, supported or unsupported metal or metal oxide containing catalysts, acid catalysts, zeolites, organometallic compounds. Examples include activated natural or synthetic material including activated, such as acid treated, natural clays such as bentonite type of synthesized silica-alumina or silica-magnesia, optionally with added oxides of zirconium, boron or thorium; mixed metal oxides supported on alumina or silica, such as tungsten-nickel sulfide or cobalt; metal and mixed metals containing catalysts such as platinum, palladium, rhenium, rhodium, copper, nickel, optionally supported on a silica or silica-alumina base; aluminum chloride, hydrogen chloride, sulfuric acid, hydrogen fluoride, phosphates, liquid phosphoric acid, phosphoric acid on kieselguhr, copper pyrophosphate pellets, phosphoric acid film on quarts, aluminosilicates, iron, vanadium, vanadium oxide on silica, nickel, silicone dioxide, carbonic anhydrase, iodine, zeolites, silver on alumina, Ziegler-natta catalysts, organometallic compounds, iron oxide stabilized by chromium oxide, copper, copper-zinc-alumina, promoted iron where the promoters can be potassium oxide, aluminum oxide, and calcium oxide, and iron-chrome.

The types of catalysts used in the catalytic process unit 224 can include, but are not limited to, natural clays, supported or unsupported metal or metal oxide containing catalysts, acid catalysts, zeolites, organometallic compounds. Examples include activated natural or synthetic material including activated, such as acid treated, natural clays such as bentonite type of synthesized silica-alumina or silica-magnesia, optionally with added oxides of zirconium, boron or thorium; mixed metal oxides supported on alumina or silica, such as tungsten-nickel sulfide or cobalt; metal and mixed metals containing catalysts such as platinum, palladium, rhenium, rhodium, copper, nickel, optionally supported on a silica or silica-alumina base; aluminum chloride, hydrogen chloride, sulfuric acid, hydrogen fluoride, phosphates, liquid phosphoric acid, phosphoric acid on kieselguhr, copper pyrophosphate pellets, phosphoric acid film on quarts, aluminosilicates, iron, vanadium, vanadium oxide on silica, nickel, silicone dioxide, carbonic anhydrase, iodine, zeolites, silver on alumina, Ziegler-natta catalysts, organometallic compounds, iron oxide stabilized by chromium oxide, copper, copper-zinc-alumina, promoted iron where the promoters can be potassium oxide, aluminum oxide, and calcium oxide, and iron-chrome.

The products of gas fermentation can be catalytically converted, for example, by catalytic process unit 224. Additionally or alternatively, the products of gas fermentation can be catalytically converted, for example, by catalytically upgrading, into molecules, or one or more second products, wherein the one or more second products are integrated into existing or newly built infrastructure or feedstock and product transportation networks. Thus, in some embodiments, molecules produced via the catalysis of the products of gas fermentation processes may also be considered desirable products or further products of fermentation. For example, in a gas fermentation system that produces ethanol, that ethanol can reacted into a range of molecules, such as propane and benzene, toluene, ethylbenzene, xylene (BTEX), and these propane and BTEX molecules can be directly introduced into the feedstock or the existing product transportation networks/pipelines.

### Ethanol and Derivatives

In one embodiment, ethanol or ethyl alcohol produced according to the method of the disclosure may be used in numerous product applications, including antiseptic hand rubs (WO 2014/100851), therapeutic treatments for methylene glycol and methanol poisoning (WO 2006/088491), as a pharmaceutical solvent for applications such as pain medication (WO 2011/034887) and oral hygiene products (U.S. Pat. No. 6,811,769), as well as an antimicrobial preservative (U.S. Patent Application No. 2013/0230609), engine fuel (U.S. Pat. No. 1,128,549), rocket fuel (U.S. Pat. No. 3,020,708), plastics, fuel cells (U.S. Pat. No. 2,405,986), home fireplace fuels (U.S. Pat. No. 4,692,168), as an industrial chemical precursor (U.S. Pat. No. 3,102,875), cannabis solvent (WO 2015/073854), as a winterization extraction solvent (WO 2017/161387), as a paint masking product (WO 1992/008555), as a paint or tincture (U.S. Pat. No. 1,408,091), purification and extraction of DNA and RNA (WO 1997/010331), and as a cooling bath for various chemical reactions (U.S. Pat. No. 2,099,090). In addition to the foregoing, the ethanol generated by the disclosed method may be used in any other application for which ethanol might otherwise be applicable.

A further embodiment comprises converting the ethanol generated by the method into ethylene. This can be accomplished by way of an acid catalyzed dehydration of ethanol to give ethylene according to the following formula:

CH₃CH₂OH→CH₂=CH₂+H₂O

The ethylene generated in this way may be used for a variety of applications on its own or can be used as a raw material for more refined chemical products. Specifically, ethylene alone may be used as an anesthetic, as part of a mixture with nitrogen to control ripening of fruit, as a fertilizer, as an element in the production of safety glass, as part of an oxy-fuel gas in metal cutting, welding and high velocity thermal spraying, and as a refrigerant.

As a raw material, ethylene can used in the manufacture of polymers such as polyethylene (PE), polyethylene terephthalate (PET) and polyvinyl chloride (PVC) as well as fibres and other organic chemicals. These products are used in a wide variety of industrial and consumer markets such as the packaging, transportation, electrical/electronic, textile and construction industries as well as consumer chemicals, coatings and adhesives.

Ethylene can be chlorinated to ethylene dichloride (EDC) and can then be cracked to make vinyl chloride monomer (VCM). Nearly all VCM is used to make polyvinyl chloride which has its main applications in the construction industry.

Other ethylene derivatives include alpha olefins which are used in Linear low-density polyethylene (LLDPE) production, detergent alcohols and plasticizer alcohols; vinyl acetate monomer (VAM) which is used in adhesives, paints, paper coatings and barrier resins; and industrial ethanol which is used as a solvent or in the manufacture of chemical intermediates such as ethyl acetate and ethyl acrylate.

Ethylene may further be used as a monomer base for the production of various polyethylene oligomers by way of coordination polymerization using metal chloride or metal oxide catalysts. The most common catalysts consist of titanium (III) chloride, the so-called Ziegler-Natta catalysts. Another common catalyst is the Phillips catalyst, prepared by depositing chromium (VI) oxide on silica.

Polyethylene oligomers so produced may be classified according to its density and branching. Further, mechanical properties depend significantly on variables such as the extent and type of branching, the crystal structure, and the molecular weight. There are several types of polyethylene which may be generated from ethylene, including, but not limited to:
Ultra-high-molecular-weight polyethylene (UHMWPE);
Ultra-low-molecular-weight polyethylene (ULMWPE or PE-WAX);
High-molecular-weight polyethylene (HMWPE);
High-density polyethylene (HDPE);
High-density cross-linked polyethylene (HDXLPE);
Cross-linked polyethylene (PEX or XLPE);
Medium-density polyethylene (MDPE);
Linear low-density polyethylene (LLDPE);
Low-density polyethylene (LDPE);
Very-low-density polyethylene (VLDPE); and
Chlorinated polyethylene (CPE).

Low density polyethylene (LDPE) and linear low-density polyethylene (LLDPE) mainly go into film applications such as food and non-food packaging, shrink and stretch film, and non-packaging uses. High density polyethylene (HDPE) is used primarily in blow molding and injection molding applications such as containers, drums, household goods, caps and pallets. HDPE can also be extruded into pipes for water, gas and irrigation, and film for refuse sacks, carrier bags and industrial lining.

According to one embodiment, the ethylene formed from the ethanol described above may be converted to ethylene oxide via direct oxidation according to the following formula:

C₂H₄+O₂→C₂H₄O

The ethylene oxide produced thereby is a key chemical intermediate in a number of commercially important processes including the manufacture of monoethylene glycol. Other EO derivatives include ethoxylates (for use in shampoo, kitchen cleaners, etc.), glycol ethers (solvents, fuels, etc.) and ethanolamines (surfactants, personal care products, etc.).

Monoethylene Glycol and Derivatives

According to one embodiment of the disclosure, the ethylene oxide produced as described above may be used to produce commercial quantities of monoethylene glycol by way of the formula:

(CH₂CH₂)O+H₂O→HOCH₂CH₂OH

According to another embodiment, the claimed microorganism can be modified in order to directly produce monoethylene glycol. As described in WO 2019/126400, the disclosure of which is incorporated by reference herein, the microorganism further comprises one or more of an enzymes capable of converting acetyl-CoA to pyruvate; an enzyme capable of converting pyruvate to oxaloacetate; an enzyme capable of converting pyruvate to malate; an enzyme capable of converting pyruvate to phosphoenolpyruvate; an enzyme capable of converting oxaloacetate to citryl-CoA; an enzyme capable of converting citryl-CoA to citrate; an enzyme capable of converting citrate to aconitate and aconitate to iso-citrate; an enzyme capable of converting phosphoenolpyruvate to oxaloacetate; an enzyme capable of converting phosphoenolpyruvate to 2-phospho-D-glycerate; an enzyme capable of converting 2-phospho-D-glycerate to 3-phospho-D-glycerate; an enzyme capable of converting 3-phospho-D-glycerate to 3-phosphonooxypyruvate; an enzyme capable of converting 3-phosphonooxypyruvate to 3-phospho-L-serine; an enzyme capable of converting 3-phospho-L-serine to serine; an enzyme capable of converting serine to glycine; an enzyme capable of converting 5,10-methylenetetrahydrofolate to glycine; an enzyme capable of converting serine to hydroxypyruvate; an enzyme capable of converting D-glycerate to hydroxypyruvate; an enzyme capable of converting malate to glyoxylate; an enzyme capable of converting glyoxylate to glycolate; an enzyme capable of converting hydroxypyruvate to glycolaldehyde; and/or an enzyme capable of converting glycolaldehyde to ethylene glycol.

Monoethylene glycol produced according to either of the described methods may be used as a component of a variety of products including as a raw material to make polyester fibers for textile applications, including nonwovens, cover stock for diapers, building materials, construction materials, road-building fabrics, filters, fiberfill, felts, transportation upholstery, paper and tape reinforcement, tents, rope and cordage, sails, fish netting, seatbelts, laundry bags, synthetic artery replacements, carpets, rugs, apparel, sheets and pillowcases, towels, curtains, draperies, bed ticking, and blankets.

MEG may be used on its own as a liquid coolant, antifreeze, preservative, dehydrating agent, drilling fluid or any combination thereof. The MEG produced may also be used to produce secondary products such as polyester resins for use in insulation materials, polyester film, de-icing fluids, heat transfer fluids, automotive antifreeze and other liquid coolants, preservatives, dehydrating agents, drilling fluids, water-based adhesives, latex paints and asphalt emulsions, electrolytic capacitors, paper, and synthetic leather.

Importantly, the monoethylene glycol produced may be converted to the polyester resin polyethylene terephthalate (“PET”) according to one of two major processes. The first process comprises transesterification of the monoethylene glycol utilizing dimethyl terephthalate, according to the following two-step process:

### First Step

C₆H₄(CO₂CH₃)₂+2HOCH₂CH₂OH→C₆H₄(CO₂CH₂CH₂OH)₂+2CH₃OH

### Second Step

nC₆H₄(CO₂CH₂CH₂OH)₂→[(CO)C₆H₄(CO₂CH₂CH₂O)]ₙ+nHOCH₂CH₂OH

Alternatively, the monoethylene glycol can be the subject of an esterification reaction utilizing terephthalic acid according to the following reaction:

nC₆H₄(CO₂H)₂+nHOCH₂CH₂OH→[(CO)C₆H₄(CO₂CH₂CH₂O)]ₙ+2nH₂O

The polyethylene terephthalate produced according to either the transesterification or esterification of monoethylene glycol has significant applicability to numerous packaging applications such as jars and, in particular, in the production of bottles, including plastic bottles. It can also be used in the production of high-strength textile fibers such as Dacron, as part of durable-press blends with other fibers such as rayon, wool, and cotton, for fiber fillings used in insulated clothing, furniture, and pillows, in artificial silk, as carpet fiber, automobile tire yarns, conveyor belts and drive belts, reinforcement for fire and garden hoses, seat belts, nonwoven fabrics for stabilizing drainage ditches, culverts, and railroad beds, and nonwovens for use as diaper topsheets, and disposable medical garments.

At a higher molecular weight, PET can be made into a high-strength plastic that can be shaped by all the common methods employed with other thermoplastics. Magnetic recording tape and photographic film are produced by extrusion of PET film. Molten PET can be blow-molded into transparent containers of high strength and rigidity that are also virtually impermeable to gas and liquid. In this form, PET has become widely used in bottles, especially plastic bottles, and in jars.

### Isopropanol and Derivatives

In an additional embodiment, isopropanol or isopropyl alcohol (IPA) produced according to the method may be used in numerous product applications, including either in isolation or as a feedstock for the production for more complex products. Isopropanol may also be used in solvents for cosmetics and personal care products, de-icers, paints and resins, food, inks, adhesives, and pharmaceuticals, including products such as medicinal tablets as well as disinfectants, sterilizers, and skin creams.

The IPA produced may be used in the extraction and purification of natural products such as vegetable and animal oil and fats. Other applications include its use as a cleaning and drying agent in the manufacture of electronic parts and metals, and as an aerosol solvent in medical and veterinary products. It can also be used as a coolant in beer manufacture, a coupling agent, a polymerization modifier, a de-icing agent and a preservative.

Alternatively, the IPA produced according to the method of the disclosure may be used to manufacture additional useful compounds, including plastics, derivative ketones such as methyl isobutyl ketone (MIBK), isopropylamines and isopropyl esters. Still further, the IPA may be converted to propylene according to the following formula:

CH₃CH₂CH₂OH→CH₃—CH═CH₂

The propylene produced may be used as a monomer base for the production of various polypropylene oligomers by way of chain-growth polymerization via either gas-phase or bulk reactor systems. The most common catalysts consist of titanium (III) chloride, the so-called Ziegler-Natta catalysts and metallocene catalysts.

Polypropylene oligomers so produced may be classified according to tacticity and can be formed into numerous products by either extrusion or molding of polypropylene pellets, including piping products, heat-resistant articles such as kettles and food containers, disposable bottles (including plastic bottles), clear bags, flooring such as rugs and mats, ropes, adhesive stickers, as well as foam polypropylene which can be used in building materials. Polypropylene may also be used for hydrophilic clothing and medical dressings.

### Commodity Chemicals and Articles

According to one embodiment, the gas fermentation product is a commodity chemical. In another embodiment, the gas fermentation product is a commodity chemical, where the commodity chemical is catalytically converted, for example, by catalytically upgrading, into molecules, or one or more second products, wherein the one or more second products are integrated into existing or newly built infrastructure or feedstock and product transportation networks. In one embodiment, wherein the commodity chemical is selected from ethanol, isopropanol, monoethylene glycol, sulfuric acid, propylene, sodium hydroxide, sodium carbonate, ammonia, benzene, acetic acid, ethylene, ethylene oxide, formaldehyde, methanol, or any combination thereof. In one embodiment, the commodity chemical is aluminum sulfate, ammonia, ammonium nitrate, ammonium sulfate, carbon black, chlorine, diammonium phosphate, monoammonium phosphate, hydrochloric acid, hydrogen fluoride, hydrogen peroxide, nitric acid, oxygen, phosphoric acid, sodium silicate, titanium dioxide, or any combination thereof. In another embodiment, the commodity chemical is acetic acid, acetone, acrylic acid, acrylonitrile, adipic acid, benzene, butadiene, butanol, caprolactam, cumene, cyclohexane, dioctyl phthalate, ethylene glycol, methanol, octanol, phenol, phthalic anhydride, polypropylene, polystyrene, polyvinyl chloride, polypropylene glycol, propylene oxide, styrene, terephthalic acid, toluene, toluene diisocyanate, urea, vinyl chloride, xylenes, or any combination thereof.

### Secondary Products

The disclosed systems and methods are also suitable for providing one or more secondary products that are independent of the gas fermentation product (e.g., ethylene, ethanol, acetate, etc.). For example, in certain embodiments, microbial biomass itself may be considered a secondary product. In such embodiments, biomass from a bioreactor, such as dead microorganisms, may be used as a carbon source for further fermentation by gasifying the biomass. Additionally or alternatively, microbial proteins or other biomass may be recovered from a bioreactor and sold/used separately from the primary product (e.g., ethylene, ethanol, acetate, 1-butanol, etc.) as a supplement, such as a nutritional supplement and/or an animal feed. Known methods for using such biomass as a nutritional supplement or animal feed are disclosed in U.S. Pat. No. 10,856,560, which is herein incorporated by reference.

Additionally or alternatively, biochar may be a secondary product. In embodiments that involve or comprise gasification of solid or liquid carbonaceous materials to produce a feedstock, biochar can be incidentally produced. Biochar is carbon rich and highly structured, and therefore it can be useful as, for example, fertilizer, among other applications.

Additionally or alternatively, unutilized carbon dioxide, which may be in the form of an off-gas from the gas fermentation, may be a secondary product. Such unutilized carbon dioxide will be in a stoichiometrically higher proportion in the off-gas compared to the feedstock, and this relative purity can make the carbon dioxide useful. For example, the unutilized carbon can be sequestered by an operator for the purposes of obtaining carbon credits, or it may be combined with hydrogen gas (H₂), such as “green hydrogen” resulting from electrolysis, and recycled back into the gas fermenter or bioreactor as feedstock.

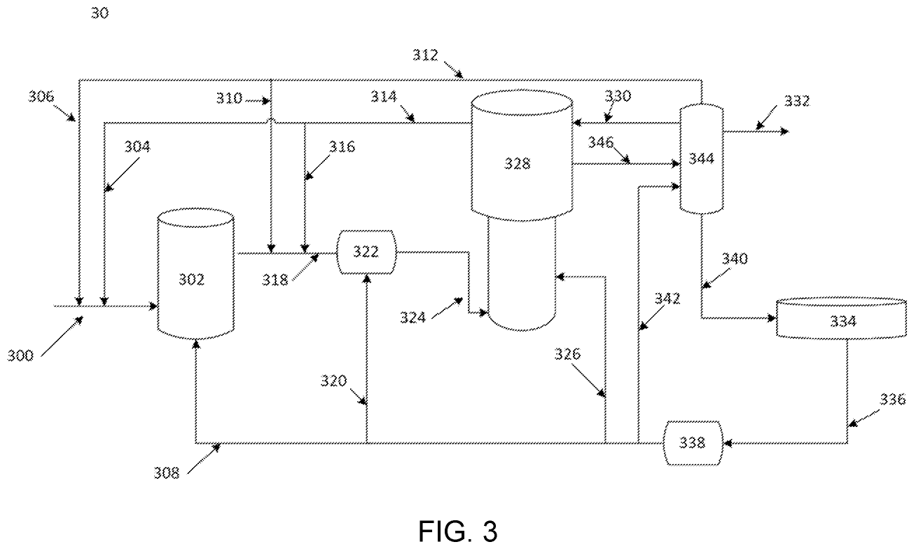

FIG. 3 shows an enlarged gas fermentation process including an optional gasification zone 302, a gas fermentation zone 328, a product recovery zone 344, and an optional wastewater treatment zone 334. Optional gasification process 302 receives a gasification feed 300, which may be any suitable material capable of being gasified to produce syngas stream 302. In various instances, gasification feed 300 may be comprised at least partially of sorted and/or unsorted industrial or municipal solid waste. In other instances, the gasification feed 300 is comprised at least partially of forest and/or agricultural waste. In particular embodiments, the gasification feed 300 is comprised of any combination of two or more of the following: sorted municipal or industrial solid waste, unsorted municipal or industrial solid waste, forest waste, agricultural waste, or other solid or liquid waste from the refining or chemical process integrated with the enlarged gas fermentation process. In another embodiment, gasification feed 300 comprises tire, pieces of tires, and or components of tires. In another embodiment gasification feed 300 comprises tires, pieces of tires, and or components of tires, in combination with an organic-based feed material. Integration internal to the enlarged fermentation process would also provide for at least one effluent from the fermentation process 328, at least one effluent from the product recovery process 344, and or at least one effluent from the wastewater treatment process 334 being used as gasification feed.

Gasification zone 302 is to produce syngas as substrate for gas fermentation zone 328. If a gas feedstock is already present for use as substrate for gas fermentation zone, such as from the refining or chemical process integrated with the enlarged gas fermentation process, gasification zone 302 may not be required. In some embodiments, syngas 318 produced by the gasification process 302, or gas obtained from another source contains one or more constituent that needs to be removed and/or converted. Typical constituents found in the syngas stream 318 that may need to be removed and/or converted include, but are not limited to, sulfur compounds, aromatic compounds, alkynes, alkenes, alkanes, olefins, nitrogen compounds, phosphorous-containing compounds, particulate matter, solids, oxygen, halogenated compounds, silicon-containing compounds, carbonyls, metals, alcohols, esters, ketones, peroxides, aldehydes, ethers, and tars. These constituents may be removed by one or more removal zones 322 positioned between gasification zone 302 and gas fermentation zone 328. Removal zone 322 may comprise one or more of the following modules: hydrolysis module, acid gas removal module, deoxygenation module, catalytic hydrogenation module, particulate removal module, chloride removal module, tar removal module, and hydrogen cyanide polishing module. Two or more modules may be combined into a single module performing the same functions. For instance, the hydrolysis module, acid gas removal module, deoxygenation module, and catalytic hydrogenation module may be combined into a single module. When incorporating removal process 322, at least a portion of the syngas 318 from gasification zone 302 is passed to removal process 322 to remove and/or convert at least a portion of at least one constituent found in syngas stream 318. Removal zone 322 may operate to bring the constituent(s) within allowable levels to produce a treated stream 324 suitable for fermentation in gas fermentation zone 328.

Gas fermentation process 328 employs at least one C1-fixing microorganism in a liquid nutrient media to ferment a feedstock, gas, or syngas stream 318 and produce one or more product. The C1-fixing microorganism in the gas fermentation process 328 may be a carboxydotrophic bacterium. In particular embodiments, the carboxydotrophic bacterium is selected from the group comprising Moorella, Clostridium, Ruminococcus, Acetobacterium, Eubacterium, Butyribacterium, Oxobacter, Methanosarcina, Methanosarcina, Cupriavidus and Desulfotomaculum. In various embodiments, the carboxydotrophic bacterium is Clostridium. In various embodiments, the carboxydotrophic bacterium is Clostridium autoethanogenum.

The one or more products produced in gas fermentation zone 328 are removed and/or separated from the fermentation broth in product recovery zone 344. Product recovery zone 344 separates and removes one or more product(s) 332 and produces at least one effluent 342, 330, 312, which comprise reduced amounts of at least one product. Product depleted effluent 342 may be passed to wastewater treatment zone 334 to produce at least one wastewater treatment zone effluent 336, which may be recycled to the gasification process 302 in line 308 and/or the fermentation process 328 in line 326.

In at least one embodiment, tail-gas effluent 314 from fermentation zone 328 is tail-gas containing gas generated by the fermentation, inert gas, and or unmetabolized substrate. At least a portion 304 of tail gas 314 may be passed to the gasification zone 302 to be used as part of the gasification feed 300. At least a portion 316 of the tail gas 314 may be passed to quench the syngas stream 318. At least a portion of the tail gas may be passed to the refinery or chemical manufacture process integrated with the enlarged gas fermentation process (not shown).

In at least one embodiment, the effluent from the fermentation zone 328 is fermentation broth 346. At least a portion of the fermentation broth 346 may be passed to product recovery zone 344. In at least one embodiment, product recovery zone 344 separates at least a portion of the microbial biomass from the fermentation broth. In various instances, at least a portion of the microbial biomass that is separated from the fermentation broth is recycled to the fermentation zone 328 via a conduit 330. In various instances, at least a portion 310 of microbial biomass-depleted water 312 that is separated from the fermentation broth 346 is recycled to the fermentation zone 328. In various instances, at least a portion 306 of the microbial biomass-depleted water 312 separated from the fermentation broth 346 is passed to optional gasification zone 302 for use as part of gasification feed 300. In certain instances, fermentation zone 328 produces fusel oil (not shown) which may also be recovered in product recovery zone 344 through any suitable means such as within the rectification column of a distillation system. In at least one embodiment, at least a portion of the fusel oil from the product recovery zone 344 is used as a heating source for one or more zones or elsewhere in the refinery or chemical process.

In various instances, at least a portion of fermentation broth 346 containing microbial biomass from fermentation zone 328 may be passed to optional gasification zone 302, without being passed to product recovery zone 344 (not shown). In various instances, at least a portion of wastewater stream 340 may be passed to optional gasification zone 302 without being passed to wastewater treatment zone 334 (not shown).

In instances where the fermentation broth is processed by the product recovery process 344, at least a portion of the microbial biomass depleted water, produced through the removal of microbial biomass from the fermentation broth, may be returned to fermentation zone 328 via a conduits 312 and 310 and/or sent via a conduits 312 and 306 to gasification zone 302. At least a portion 306 of the microbial biomass depleted water 312 may be passed to gasification zone 302 to be used as part of gasification feed 300. At least a portion 310 of the microbial biomass depleted water 312 may be passed to quench syngas stream 318. At least a portion of the effluent from product recovery zone 344 may be passed via a conduit 342 to wastewater treatment zone 334. The effluents from product recovery zone 344 may comprise reduced amounts of product and/or microbial biomass.

Wastewater treatment zone 334 receives and treats effluent from one or more zones to produce clarified water. The clarified water may be passed or recycled via a conduit 336 to one or more zones. For example, at least a portion 326 of the clarified water 336 may be passed to the fermentation zone 328, at least a portion 308 of the clarified water 336 may be passed to gasification zone 302 to be used as part of the gasification feed 300 and at least a portion 320 of the clarified water 336 may be passed to quench syngas stream 318. In certain instances, the wastewater treatment process 334 generates biomass as part of the treatment process. At least a portion of this biomass may be passed via conduit 308 to the gasification zone 302 for use as part of gasification feed 300. Wastewater treatment zone 334, as a by-product of treating microbial biomass, may produce biogas. At least a portion of the biogas may be passed via conduit 308 to gasification zone 302 to be used as part of gasification feed 300 and or via a conduit 320 to quench syngas stream 318.

Optional wastewater treatment effluent removal unit 338 is positioned downstream of wastewater treatment zone 334. At least a portion of biogas from wastewater treatment zone 334 is passed to removal unit 338 to remove and/or convert at least a portion of at least one constituent found in the biogas stream. Removal unit 338 operates to lower the concentration of constituents to within preterminal allowable levels and produce a treated stream 342, 326, 320, and/or 308 suitable to be used by the subsequent one or more zones 344, 328, 322, and/or 302, respectively.

According to an embodiment, a GF process is integrated at an oil refining site, and biologically produces a gas fermentation product. The GF process would be co-located within the operating site of the oil refiner. Underutilized carbon streams (e.g., waste carbon streams) from various points within the operating site could be redirected to feed the GF process to produce various gas fermentation products. Similarly, a product stream from the GF process may be integrated at an oil refining or chemical manufacturing site.

According to the embodiment, integration of GF units with existing facilities and infrastructure provides multitude of advantageous synergies for operators. These synergies include using the gas from one portion of a chemical manufacturing unit as the feed for the GF unit. The GF unit being adapted to convert the gas feed into a product that the same unit is overall designed to produce. The GF unit's output can then be directed to a portion of the unit that can recover the product from the GF unit's output. Thereby increasing the overall production yield and efficiency of the complex by converting previously designated waste or underutilized carbon into marketable product.

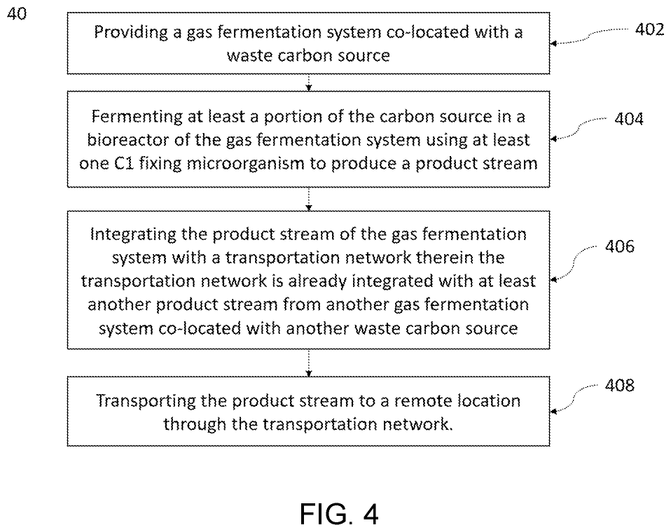

FIG. 4 illustrates an embodiment for a method of operation of an integrated gas fermentation unit. The method includes the steps of first 402 providing a gas fermentation system co-located with a carbon source (e.g., a waste carbon source). Then at step 404, fermenting at least a portion of the carbon source in a bioreactor of the gas fermentation system using at least one C1 fixing microorganism to produce a product stream. Then at step 406, integrating the product stream of the gas fermentation system with a transportation network where the transportation network is already integrated with at least another product stream from another gas fermentation system co-located with another carbon source. Finally at step 408, transporting the product stream to a remote location through the transportation network.

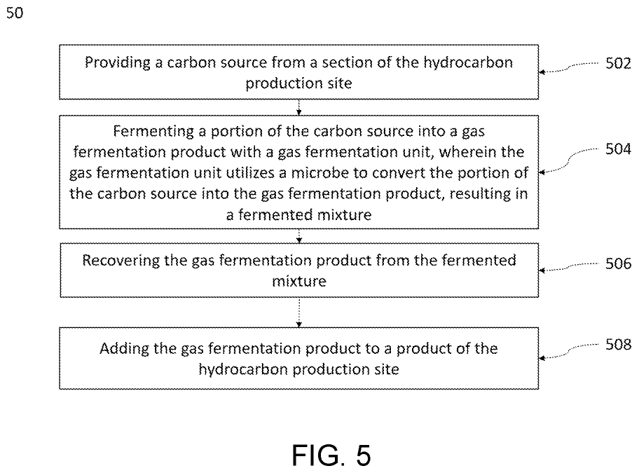

FIG. 5 illustrates an embodiment for a method of operation of an integrated gas fermentation unit. The method includes the steps of first 502 providing a carbon source from a section of the chemical manufacturing or oil refining complex 100. Then at step 504, fermenting a portion of the carbon source into a gas fermentation product with a gas fermentation unit, wherein the gas fermentation unit utilizes a microorganism to convert the portion of the carbon source into the gas fermentation product, resulting in a fermented mixture. Then at step 506, recovering the gas fermentation product from the fermented mixture. Finally at step 508, adding the gas fermentation product to a product of the chemical manufacturing or oil refining complex 100.

In another embodiment, a GF unit that is integrated within a chemical manufacturing complex or oil refining complex can have its output directly coupled to a separation, purification, or extraction unit. The separation, purification, or extraction unit being adapted to receive the output of the GF unit and separate or purify the output mixture. The recovered product can then be routed to a product storage tank located within the chemical manufacturing or oil-refining complex to be sold. In other embodiments, the recovered product can be mixed with other products as an additive to increase desirable properties of the base product or to decrease the effect of undesirable properties of the base product. In other embodiments, the recovered product can be stored separately and sold as a standalone product.

In another embodiment, a GF unit that is integrated within a chemical manufacturing complex or oil refining complex can have its output directly utilized by a process of the chemical manufacturing operation or refinery. The product of the GF process, for example, may be a reactant needed for an existing operation, it may be a solvent needed for an existing operation, it may be an extractant for an existing operation, and the like. The existing process of the chemical manufacturing operation or the refinery being adapted to receive the output of the GF unit and employ the product for a needed function. The resulting overall product may have a greater portion attributable to sustainable sources as compared to the same product without the integration of gas fermentation.

According to another embodiment, GF unit's integrated within chemical manufacturing complexes can be adapted to convert gases as described above into fuel for various operationally critical components such as furnaces, boilers, co-generation units, or power generation units. The converted gases can then be routed from the GF unit to the feed lines for the aforementioned components to supplement or replace the fuel feed that would otherwise be derived from fossil carbon sources such as natural gas. This synergy allows the operator to lower their overall spend on external fuel sources to continue operations. Additionally, this synergy allows operators to be more energy independent and less dependent on external fuel supply fluctuations caused by market activity or natural disasters such as hurricanes.

In other embodiments, the GF unit is adapted to output to an on-site storage tank. The on-site storage tank can then accept the GF unit's output and store the output until the price of the product contained in the GF unit's output mixture is sufficiently high such that it becomes desirable for the production operator to sell the product. In yet another embodiment, the storage tank or the GF unit output can each have a metering flow meter installed therein to determine the amount of product that is exiting the output of the GF unit or the product storage tank. Metering can be done with any suitable metering device; non-limiting examples include a mass flow meter and a volumetric flow meter.

In yet another embodiment, multiple GF units can be integrated at a single chemical manufacturing or oil-refining complex wherein each GF unit is configured to accept the same or different types of gas sources. This can be done such that each GF unit can be individually adapted to output a different product in its respective output mixture. Varying the input streams to each GF unit can be done to increase the feed to GF units adapted to produce products that are contemporaneously more profitable than others. Likewise, the feed to contemporaneously less profitable products can be throttled down or halted completely. The various outputs from the various GF units can be configured such to be routed to individual storage tanks for short, medium, or long term storage or routed directly into a feed source for other sections of the complex.

In another embodiment, the GF unit can be configured to produce fuels that can be utilized on-site by the chemical manufacturing or oil-refining complex. Such synergy allows for the complex to lower the overall operating costs by decreasing the spend related to such fuels and fuel sources, thereby increasing the overall profitability of the complex.

In yet another embodiment, the GF unit can be fed from pressure relief safety valves (“PRSV”). A PRSV is typically installed on a pressure containing vessel and is adapted to open at a set pressure value to protect the vessel it is connected to. Typically, the streams contained within the pressure vessel are carbon rich and are routed to a safe relief point, either a flare source or the atmosphere. Operators can further increase their site productivity and efficiency by harvesting these otherwise wasted streams of carbon rich material (i.e., any gas stream that is either emitted directly, flared with no additional value capture or combusted for energy recovery purposes) by routing them to the integrated GF unit to be converted into marketable products. In other words, these otherwise wasted streams can be used as a feedstock for the integrated GF, which can upgrade the C1 components of the underutilized carbon into desirable products or chemicals with two or more carbon-carbon bonds.

In other embodiments, multiple GF units can be integrated within a single chemical manufacturing or oil-refining complex. The multiple GF units can be adapted to produce a similar or identical product from various different input streams. The multiple GF units can then have their respective outputs configured to output to a single storage tank. This synergy can allow the production operator to capture a maximum amount of underutilized gases or streams and convert it into a marketable product.

### D. MICROORGANISMS AND FERMENTATION

The disclosed systems and methods integrate microbial fermentation into existing or newly built infrastructure of, for example, a gas (e.g., natural gas) transportation pipeline, oil well, or the like to convert various feedstocks, gas, or other by-products into useful products such as ethylene. As disclosed herein, the systems allow for feedstocks, gas, or other by-products to be directly provided to a bioreactor, and the bioreactor is directly connected to a system for facilitating transport of a desirable product of fermentation to an end point (e.g., a chemical plant or refinery). In particular, the disclosed systems and methods are applicable for producing useful products (e.g., ethylene, ethanol, acetate, etc.) from gaseous substrates, such as gases that may optionally contain H₂, that are utilized as a carbon source by microbial cultures. Such microorganisms may include bacteria, archaea, algae, or fungi (e.g., yeast), and these classes of microorganism may be suitable for the disclosed systems and methods. In general, the selection of the microorganism(s) is not particularly limited so long as the microorganism is C1-fixing, carboxydotrophic, acetogenic, methanogenic, capable of Wood-Ljundahl synthesis, a hydrogen oxidizer, autotrophic, chemolithoautotrophic, or any combination thereof. Among the various suitable classes of microorganisms, bacteria are particularly well suited for integration in the disclosed systems and methods.

When bacteria are utilized in the disclosed systems and methods, the bacteria may be aerobic or anaerobic, depending on the nature of the carbon source and other inputs being fed into the bioreactor or fermentation unit. Further, the bacteria utilized in the disclosed systems and methods can include one of more strains of carboxydotrophic bacteria. In particular embodiments, the carboxydotrophic bacterium can be selected from a genus including, but not limited to, Cupriavidus, Clostridium, Moorella, Carboxydothermus, Ruminococcus, Acetobacterium, Eubacterium, Butyribacterium, Oxobacter, Methanosarcina, Methanosarcina, and Desulfotomaculum. In particular embodiments, the carboxydotrophic bacterium is Clostridium. In particular embodiments, the carboxydotrophic bacterium is Clostridium autoethanogenum. In particular embodiments, the carboxydotrophic bacterium is Cupriavidus. In other particular embodiments, the carboxydotrophic bacterium is Cupriavidus necator.

A number of anaerobic bacteria are known to be capable of carrying out fermentation for the disclosed methods and system. Examples of such bacteria that are suitable for use in the invention include bacteria of the genus Clostridium, such as strains of Clostridium ljungdahlii (including those described in WO 00/68407, EP 117309, U.S. Pat. Nos. 5,173,429, 5,593,886, and 6,368,819, WO 98/00558 and WO 02/08438), Clostridium carboxydivorans (Liou et al., International Journal of Systematic and Evolutionary Microbiology 33: pp 2085-2091) and Clostridium autoethanogenum (Abrini et al., Archives of Microbiology 161: pp 345-351). Other suitable bacteria include those of the genus Moorella, including Moorella sp HUC22-1 (Sakai et al., (2004) Biotechnology Letters 26: pp 1607-1612), and those of the genus Carboxydothermus (Svetlichny, V. A., et al. (1991), Systematic and Applied Microbiology 14: 254-260). The disclosures of each of these publications are incorporated herein by reference. In addition, other carboxydotrophic anaerobic bacteria can be used in the disclosed systems and methods by a person of skill in the art. It will also be appreciated upon consideration of the instant disclosure that a mixed culture of two or more bacteria may be used in in the disclosed systems and methods. All of the foregoing patents, patent applications, and non-patent literature are incorporated herein by reference in their entirety.

One exemplary anaerobic bacteria that is suitable for use in the disclosed systems and methods is Clostridium. One exemplary anaerobic bacteria that is suitable for use in the disclosed systems and methods is Clostridium autoethanogenum. In some embodiments, the Clostridium autoethanogenum is a Clostridium autoethanogenum having the identifying characteristics of the strain deposited at the German Resource Centre for Biological Material (DSMZ) under the identifying deposit number 19630. In some embodiments, the Clostridium autoethanogenum is a Clostridium autoethanogenum having the identifying characteristics of DSMZ deposit number DSMZ 10061. In some embodiments, the Clostridium autoethanogenum is a Clostridium autoethanogenum having the identifying characteristics of DSMZ deposit number DSMZ 23693.

In some embodiments, the anaerobic bacteria is Clostridium carboxidivorans having the identifying characteristics of deposit number DSM15243. In some embodiments, the anaerobic bacteria is Clostridium drakei having the identifying characteristics of deposit number DSM12750. In some embodiments, the anaerobic bacteria is Clostridium ljungdahlii having the identifying characteristics of deposit number DSM13528. Other suitable Clostridium ljungdahlii strains may include those described in WO 00/68407, EP 117309, U.S. Pat. Nos. 5,173,429, 5,593,886, and 6,368,819, WO 98/00558 and WO 02/08438, all of which are incorporated herein by reference. In some embodiments, the anaerobic bacteria is Clostridium scatologenes having the identifying characteristics of deposit number DSM757. In some embodiments, the anaerobic bacteria is Clostridium ragsdaleii having the identifying characteristics of deposit number ATCC BAA-622.

In some embodiments, the anaerobic bacteria is Acetobacterium woodii. In some embodiments, the anaerobic bacteria is from the genus Moorella, such as Moorella sp HUC22-1, (Sakai et al, (2004) Biotechnology Letters, 26: pp 1607-1612). Further examples of suitable anaerobic bacteria include, but are not limited to, Morella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp 41-65). In addition, it should be understood that other C1-fixing, carboxydotrophic anaerobes may be suitable for the disclosed systems and methods. It will also be appreciated that a mixed culture of two or more bacteria may be utilized as well.

A number of aerobic bacteria are known to be capable of carrying out fermentation for the disclosed methods and system. Examples of such bacteria that are suitable for use in the invention include bacteria of the genus Cupriavidus and Ralstonia. In some embodiments, the aerobic bacteria is Cupriavidus necator or Ralstonia eutropha. In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantogensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

The fermentation may be carried out in any suitable bioreactor. In some embodiments, the bioreactor may comprise a first, growth reactor in which the microorganisms (e.g., bacteria) are cultured, and a second, fermentation reactor, to which fermentation broth from the growth reactor is fed and in which most of the fermentation product (e.g. ethylene, ethanol, acetate, etc.) is produced.

It will be appreciated that for growth of the bacteria and fermentation to occur, in addition to a carbon-containing substrate gas, a suitable liquid nutrient medium will need to be fed to the bioreactor. A nutrient medium will contain vitamins and minerals sufficient to permit growth of the micro-organism used. Aerobic and anaerobic media suitable for the fermentation using carbon-containing substrate gases as the sole carbon source are known in the art. For example, suitable media are described in U.S. Pat. Nos. 5,173,429, 5,593,886, WO 02/08438, WO2007/115157, and WO2008/115080, referred to above and all of which are incorporated herein by reference. Further, the fermentation can be carried out under appropriate conditions for the desired fermentation to occur. Reaction conditions that should be considered include pressure, temperature, gas flow rate, liquid flow rate, media pH, media redox potential, agitation rate (if using a continuous stirred tank reactor), inoculum level, maximum gas substrate concentrations, and maximum product concentrations to avoid product inhibition.

The optimum reaction conditions will depend partly on the particular micro-organism used. However, in general, it may be preferable that the fermentation be performed at a pressure higher than ambient pressure. Operating at increased pressures may allow for, for example, a significant increase in the rate of CO transfer from the gas phase to the liquid phase where it can be taken up by the micro-organism as a carbon source. This, in turn, means that the retention time (defined as the liquid volume in the bioreactor divided by the input gas flow rate) can be reduced when bioreactors are maintained at elevated pressure rather than atmospheric pressure. Also, since a given CO, or CO₂ and H₂ conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment.

Similarly, the temperature of the culture may vary as needed. For example, in some embodiments, the fermentation is carried out at a temperature of about 34° C. to about 37° C. In some embodiments, the fermentation is carried out at a temperature of about 34° C. This temperature range may assist in supporting or increasing the efficiency of fermentation including, for example, maintaining or increasing the growth rate of bacteria, extending the period of growth of bacteria, maintaining or increasing production of the desired product (e.g., ethylene, ethanol, acetate, etc.), or maintaining or increasing CO or CO₂ uptake or consumption.

Culturing of the bacteria used in in the disclosed systems and methods may be conducted using any number of processes known in the art for culturing and fermenting substrates. In some embodiments a culture of a bacterium can be maintained in an aqueous culture medium. For example, the aqueous culture medium may be a minimal anaerobic microbial growth medium. Suitable media are known in the art and described for example in U.S. Pat. Nos. 5,173,429 and 5,593,886; WO 02/08438, and in Klasson et al (1992), Bioconversion of Synthesis Gas into Liquid or Gaseous Fuels, Enz. Microb. Technol. 14:602-608; Najafpour and Younesi (2006) Ethanol and acetate synthesis from waste gas using batch culture of Clostridium ljungdahlii. Enzyme and Microbial Technology, 38(1-2):223-228; and Lewis et al., (2002), Making the connection: conversion of biomass-generated producer gas to ethanol, Proceedings Bioenergy 2002 Conference, p. 1-8.

Further general processes for using gaseous substrates for fermentation that may be utilized for the disclosed systems and methods are described in the following disclosures: WO98/00558, M. Demler and D. Weuster-Botz (2010), Reaction Engineering Analysis of Hydrogenotrophic Production of Acetic Acid by Acetobacterium woodii, Biotechnology and Bioengineering; D. R. Martin, A. Misra and H. L. Drake (1985), Dissimilation of Carbon Monoxide to Acetic Acid by Glucose-Limited Cultures of Clostridium thermoaceticum, Applied and Environmental Microbiology, 49(6):1412-1417. Further processes generally described in the following articles using gaseous substrates for fermentation may also be utilized: (i) K. T. Klasson, et al. (1991), Bioreactors for synthesis gas fermentations resources, Conservation and Recycling, 5:145-165; (ii) K. T. Klasson, et al. (1991), Bioreactor design for synthesis gas fermentations, Fuel, 70:605-614; (iii) K. T. Klasson, et al. (1992), Bioconversion of synthesis gas into liquid or gaseous fuels, Enzyme and Microbial Technology, 14:602-608; (iv) J. L. Vega, et al. (1989), Study of Gaseous Substrate Fermentation: Carbon Monoxide Conversion to Acetate. 2. Continuous Culture, Biotech. Bioeng., 34(6):785-793; (vi) J. L. Vega, et al. (1989), Study of gaseous substrate fermentations: Carbon monoxide conversion to acetate. 1. Batch culture, Biotech. Bioeng., 34(6):774-784; (vii) J. L. Vega, et al. (1990), Design of Bioreactors for Coal Synthesis Gas Fermentations, Resources, Conservation and Recycling, 3:149-160; all of which are incorporated herein by reference.

As noted above, while bacteria may be preferred microorganisms for the disclosed systems and methods, other microorganisms like yeast may also be suitable. For example, yeast that may be used in the disclosed systems and methods include genus Cryptococcus, such as strains of Cryptococcus curvatus (also known as Candida curvatus) (see Chi et al. (2011), Oleaginous yeast Cryptococcus curvatus culture with dark fermentation hydrogen production effluent as feedstock for microbial lipid production, International Journal of Hydrogen Energy, 36:9542-9550, which is incorporated herein by reference). Other suitable yeasts include those of the genera Candida, Lipomyces, Rhodosporidium, Rhodotorula, Saccharomyces, and Yarrowia. In addition, it should be understood that the disclosed systems and methods may utilize a mixed culture of two or more yeasts. Additional fungi that may be suitable for the disclosed systems and methods include, but are not limited to, fungi selected from Blakeslea, Cryptococcus, Cunninghamella, Mortierella, Mucor, Phycomyces, Pythium, Thraustochytrium and Trichosporon. Culturing of yeast or other fungi may be conducted using any number of processes known in the art for culturing and fermenting substrates using yeasts or fungi.

Typically, fermentation is carried out in any suitable bioreactor, such as a continuous stirred tank reactor (CTSR), a bubble column reactor (BCR) or a trickle bed reactor (TBR). Also, in some embodiments, the bioreactor may comprise a first, growth reactor in which the micro-organisms are cultured, and a second, fermentation reactor, to which fermentation broth from the growth reactor is fed and in which most of the fermentation product (e.g., ethylene, ethanol, acetate, etc.) is produced.

The disclosed systems and method may comprise a primary bioreactor and a secondary bioreactor. The efficiency of the fermentation processes may be further improved by a further process of recycling a stream exiting the secondary bioreactor to at least one primary reactor. The stream exiting the secondary bioreactor may contain unused substrates, salts, and other nutrient components. By recycling the exit stream to a primary reactor, the cost of providing a continuous nutrient media to the primary reactor can be reduced. This recycling step has the further benefit of potentially reducing the water requirements of the continuous fermentation process. The stream exiting the bioreactor can optionally be treated before being passed back to a primary reactor. For example, because yeasts generally require oxygen for growth, any media recycled from a secondary bioreactor to a primary bioreactor may need to have all oxygen substantially removed, as any oxygen present in the primary bioreactor will be harmful to an anaerobic culture in the primary bioreactor. Therefore, the broth stream exiting the secondary bioreactor may be passed through an oxygen scrubber to remove substantially all of the oxygen prior to being passed to the primary reactor. In some embodiments, biomass from a bioreactor (e.g., a primary bioreactor, secondary bioreactor, or any combination thereof) may be separated and processed to recover one or more products.

In some embodiments, both anaerobic and aerobic gases can be used to feed separate cultures (e.g., an anaerobic culture and an aerobic culture) in two or more different bioreactors that are both integrated into the same process stream.

As disclosed herein, the feedstock gas stream providing a carbon source for the disclosed cultures is not particularly limited, so long as it contains a carbon source. C1 feedstocks comprising methane, carbon monoxide, carbon dioxide, or any combination thereof may be preferred. Optionally, H₂ may also be present in the feedstock. In some embodiments, the feedstock may comprise a gaseous substrates comprising substrate comprising carbon monoxide. In some embodiments, the feedstock may comprise a gaseous substrates comprising substrate comprising carbon dioxide. In some embodiments, the feedstock may comprise a gaseous substrates comprising substrate comprising both carbon monoxide and carbon dioxide. In some embodiments, the feedstock may comprise a gaseous substrates comprising carbon monoxide. In some embodiments, the feedstock may comprise a gaseous substrates comprising carbon dioxide. In some embodiments, the feedstock may comprise a gaseous substrates comprising carbon monoxide, carbon dioxide, or any combination thereof.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

The microorganisms of the disclosure may be cultured with the gaseous substrate to produce one or more products. For instance, products of interest for the disclosed systems and methods can include, but are not limited to, alcohols, acids, diacids, alkanes, alkenes, alkynes, and the like. For instance, a product of interest for the disclosed systems and method can include alcohols such as ethanol. More specifically, the microorganisms of the present disclosure may produce or may be engineered to produce ethylene (WO 2012/026833, US 2013/0157322), ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611, and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US 2012/0045807), butadiene (WO 2012/024522, US 2012/0045807), methyl ethyl ketone (2-butanone) (WO 2012/024522, US 2012/0045807 and WO 2013/185123, U.S. Pat. No. 9,890,384), acetone (WO 2012/115527, U.S. Pat. No. 9,410,130), isopropanol (WO 2012/115527, U.S. Pat. No. 9,410,130), lipids (WO 2013/036147, U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567, U.S. Pat. No. 9,347,076), 2-butanol (WO 2013/185123, U.S. Pat. No. 9,890,384), 1,2-propanediol (WO 2014/036152, U.S. Pat. No. 9,284,564), 1propanol (WO 2017/066498, U.S. Pat. No. 9,738,875), 1hexanol (WO 2017/066498, U.S. Pat. No. 9,738,875), 1octanol (WO 2017/066498, U.S. Pat. No. 9,738,875), chorismate-derived products (WO 2016/191625, U.S. Pat. No. 10,174,303), 3hydroxybutyrate (WO 2017/066498, U.S. Pat. No. 9,738,875), 1,3butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and monoethylene glycol (WO 2019/126400, U.S. Pat. No. 11,555,209), or any combination thereof. For example, in some embodiments, the microorganisms may produce or may be engineered to produce one or more of the foregoing products (e.g., ethanol, acetate, 1-butanol, etc.) in addition to ethylene.

### Substrates and C1-Carbon Sources

The substrate and/or C1-carbon source may be a gas obtained as a by-product of an industrial process or from another source, such as combustion engine exhaust fumes, biogas, landfill gas, direct air capture, flaring, or from electrolysis. The substrate and/or C1-carbon source may be syngas generated by pyrolysis, torrefaction, or gasification. In other words, carbon in solid or liquid materials may be recycled by pyrolysis, torrefaction, or gasification to generate syngas which is used as the substrate and/or C1-carbon source in gas fermentation. The substrate and/or C1-carbon source may be natural gas. The substrate and/or C1-carbon source carbon dioxide from conventional and unconventional gas production. The substrate and/or C1-carbon source may be a gas comprising methane. Gas fermentation processes are flexible and any of these substrate and/or C1-carbon sources may be employed.

In certain embodiments, the industrial process source of the substrate and/or C1 carbon source is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, landfill operations, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. Other general examples include flue gas from fired boilers and fired heaters, such as natural gas, oil, or coal fired boilers or heaters, and gas turbine exhaust. Another example is the flaring of compounds such as at oil and gas production sites. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, plasma, or gasification processes. Examples of gasification processes include gasification of coal, gasification of refinery residues, gasification of petroleum coke, gasification of biomass, gasification of lignocellulosic material, gasification of wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material, and/or gasification of tires, pieces of tires, and or components of tires or tires, pieces of tires, and or components of tires in combination with an organic material. Examples of reforming processes include, steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibers such as in shoes, apparel, and textiles. Municipal solid waste may be simply landfill-type waste and may be sorted or unsorted. Examples of biomass may include lignocellulosic material and microbial biomass. Lignocellulosic material may include agriculture by-products, forest by-products, and some industrial by-products.

Biomass may be created as by-products of “nature-based solutions” (NBS) and thus natured-based solutions may provide feedstock to the gas fermentation process. Nature-based solutions is articulated by the European Commission as solutions inspired and supported by nature, which are cost-effective, simultaneously provide environmental, social, and economic benefits and help build resilience. Such solutions bring more, and more diverse, nature and natural features and processes into cities, landscapes, and seascapes, through locally adapted, resource-efficient, and systemic interventions. Nature-based solutions must benefit biodiversity and support the delivery of a range of ecosystem services. Through the use of NBS healthy, resilient, and diverse ecosystems (whether natural, managed, or newly created) can provide solutions for the benefit of both societies and overall biodiversity. Examples of nature-based solutions include natural climate solutions (conservation, restoration and improved land management that increase carbon storage or avoid greenhouse gas emissions in landscapes and wetlands across the globe), halting biodiversity loss, socio-economic impact efforts, habitat restoration, and health and wellness efforts with respect to air and water. Biomass produced through nature-based solutions may be used as feedstock to gas fermentation processes.

As shown, the optional step of a gasification process in the overall gas fermentation process greatly increases suitable feedstocks to the overall gas fermentation process as compared to gaseous feedstocks alone. Further, incentives achieved may extend beyond items such as carbon credits, and into the natural based solutions space.

The substrate and/or C1-carbon source may be a gas stream comprising methane. Such a methane containing gas may be obtained from: fossil methane emissions such as during fracking, wastewater treatment, livestock, agriculture, and municipal solid waste landfills. It is also envisioned that the methane may be burned to produce electricity or heat and the C1 by-products may be used as the substrate or carbon source. The substrate and/or C1-carbon source may be a gas stream comprising natural gas.

### E. EXAMPLES

The following examples are given to illustrate the present disclosure. It should be understood that the invention is not to be limited to the specific conditions or details described in these examples.

### Example 1—Conversion of Organic Acid to Corresponding Alcohol

### Example 1A Conversion of Butyric Acid to Butanol in a CSTR

An eight-liter reactor was filled with 7200 ml of the media LM23 and autoclaved for 30 minutes at 121° C. While cooling down, the media was sparged with N₂. The gas was switched to 95% CO, 5% CO₂ prior inoculation with 160 ml of a Clostridium autoethanogenum culture. The bioreactor was maintained at 37° C. stirred at 200 rpm at the start of the culture. During the growth phase, the agitation was increased to 500 rpm. The pH was set to 5.5 and maintained by automatic addition of 5 M NaOH. The n-butyrate solution containing 20 g butyric acid buffered to pH 5.5 was added directly into the actively growing culture. Samples of the fermentation broth were taken at 0, 24 and 48 hours after butyric acid addition (see Table 1).

TABLE 1
Conversion of 20 g of n-Butyrate into 1-Butanol by an
8 litre culture of C. autoethanogenum producing acetate
and ethanol in a bioreactor maintained at pH 5.5.

| Time [h] | 0 | 24 | 48 |
|---|---|---|---|
| Butanol | 0.0 | 4.0 | 8.2 |
| produced [g] | | | |

Starting conditions: active culture of C. autoethanogenum, producing acetate (8.3 g/l) and ethanol (5.4 g/l) pH 5.5 and sparging gas containing 95% CO in CO₂.

### Example 2—Conversion of Acetate and Butyrate to Corresponding Alcohols

Serum vials were prepared in accordance with the above. Once microbial growth was established (associated with acetate and small amounts of ethanol produced), the following compounds were added into the 50 ml active culture in the serum bottle: 1 ml of Sodium Dithionite 10 g/l solution, 2 ml of n-butyric acid solution 100 g/l (pH adjusted to 5.5 with Sodium Hydroxide 5 M). The gas phase was exchanged for 25 psig overpressure of a mixture of 95% CO, 5% CO₂ gas. After addition of the acid, 1 ml sample was taken for quantification of the metabolites at various time points (see Table 2)

TABLE 2
Conversion of n-butyrate into 1-Butanol by a culture
of C. autoethanogenum producing acetate and
ethanol in a serum bottle at pH 5.5 in presence
or absence of 0.8 mM methylviologen (MV).

| | Methyl- | | | | |
|---|---|---|---|---|---|
| Time | viologen | Acetate | Ethanol | Butyrate | Butanol |
| (h) | conc (mM) | conc (g/L) | conc (g/L) | conc (g/L) | conc (g/L) |
| 0 | 0 | 4.50 | 1.26 | 4.19 | 0.00 |
| 2 | 0 | 5.00 | 1.40 | 3.93 | 0.16 |
| 4 | 0 | 4.19 | 1.28 | 3.68 | 0.30 |
| 22 | 0 | 4.61 | 1.44 | 3.81 | 0.41 |
| 0 | 0.8 | 4.72 | 1.24 | 3.81 | 0.00 |
| 2 | 0.8 | 4.87 | 1.42 | 3.66 | 0.17 |
| 4 | 0.8 | 4.88 | 1.49 | 3.42 | 0.34 |
| 22 | 0.8 | 4.15 | 1.84 | 2.27 | 1.35 |

Starting conditions: active culture of C. autoethanogenum, producing acetate (4.7 g/l) and ethanol (1.2 g/l) pH 5.5, headspace: 25 psig overpressure of 95% CO in CO₂.

The presence of the mediator methyl viologen significantly inhibits conversion of n-butyrate to n-butanol (Table 2).

The results illustrate a number of significant advantages over previously reported methods for the microbial conversion of acids to their corresponding alcohols. For example, it demonstrated that Clostridium autoethanogenum can be used to produce alcohols which it was not known to be able to produce under standard fermentation conditions.

The bacterial cells do not need to be harvested prior to addition of acid to produce a desired alcohol; the conversion of acid to alcohol is carried out directly in the culture media. This significantly reduces handling of cells, the risk of cell damage which may be caused by centrifugation and resuspension, and the risk of oxygen contamination.

The conversion does not require the use of a mediator, such as methyl viologen. In fact, the addition of methyl viologen was demonstrated to inhibit or at least reduce the rate of conversion of acids to alcohols. Such mediators are often toxic. Removing the need for a mediator has the advantage of reducing handling of toxic chemicals and reducing the costs associated with production of alcohols.

At least in the case of C. autoethanogenum, the bacterial cells can be maintained at the same pH and temperature during the growth phase and the acid to alcohol conversion phase (37° C. and pH 5.5). This simplifies the process and reduces the risk of shock to the cells.

Further, the addition of the acid when the bacteria are in the conversion phase, and the ability of the cells to continue to consume carbon monoxide and produce acetate and ethanol (for example) while they are converting the added acids to corresponding alcohols, provides a method in which a number of valuable products can be produced simultaneously.

### Example 3—Conversion of Various Acids to Corresponding Alcohols

Serum vials were prepared in accordance with the above. However, 5 mL of an aqueous acid solution was added to the empty vial and the pH adjusted to 5.5 with NaOH. Sodium dithionate (0.5 mL of a 10 g/L aqueous solution) or cysteine (1 mL of a 6.25 g/L aqueous solution) was added prior to inoculation. Each serum vial was pressurized to 30 psig with 95% CO gas and incubated at 37° C. with constant shaking. Samples of the fermentation broth were taken at 72 h (see Table 3).

TABLE 3
Conversion of various acids to corresponding
alcohols by Clostridium autoethanogenum.

| | | Initial acid | Alcohol | |
|---|---|---|---|---|
| Reducing | | concentration | concentration | |
| agent | Acid | (g/L) | at 72 h (g/L) | Alcohol |
| Sodium | Propionic | 0.9 | 0.14 | (propanol) |
| dithionite | | | | |
| Sodium | Propionic | 1.4 | 0.38 | (propanol) |
| dithionite | | | | |
| Sodium | Butyric | 2.3 | 0.17 | (butanol) |
| dithionite | | | | |
| Sodium | Butyric | 3.1 | 0.52 | (butanol) |
| dithionite | | | | |
| Sodium | Valeric | 1.0 | 0.14 | (pentanol) |
| dithionite | | | | |
| Sodium | Valeric | 1.7 | 0.24 | (pentanol) |
| dithionite | | | | |
| Sodium | Hexanoic | 0.9 | 0.06 | (hexanol) |
| dithionite | | | | |
| Sodium | Hexanoic | 1.7 | 0.09 | (hexanol) |
| dithionite | | | | |
| Cysteine | Isovaleric | 1.41 | 0.03 | (3- |
| | | | | methylbutanol) |
| Cysteine | 2-methyl- | 1.87 | 0.06 | (2- |
| | butyric | | | methylbutanol) |

As can be seen above, Clostridium autoethanogenum can be used to convert a variety of acids to their corresponding alcohols in the presence of a reducing agent. Again, this is particularly significant, as the above acids and alcohols are not known to be naturally produced metabolites of C. auto.

### Example 4—Effect of CO Partial Pressure

### Example 4A—Effect of the CO Partial Pressure on Alcohol Production

Serum vials were prepared in accordance with the above. Each serum vial was pressurized to 30 or 40 or 50 psia using a 95% CO in CO₂ gas mixture and incubated at 37° C. with constant shaking. Samples of the fermentation broth were taken at 18 h (see Table 4)

TABLE 4
Effect of different headspace overpressures on the metabolism
of a gaseous substrate comprising 95% CO in CO₂ by a culture
of C. autoethanogenum in a serum bottle at pH 5.5
after 18 hours of fermentation.

| | Start Overpressure | | |
|---|---|---|---|
| | 30 psia | 40 psia | 50 psia |
| Acetate (g/l) | 5.4 | 5.7 | 0.2 |
| Ethanol (g/l) | 0.5 | 0.8 | 2.6 |
| Final | 2 | 8 | 29 |
| Overpressure | | | |
| (psig) | | | |
| Pressure drop | 13 | 17 | 6 |
| (psi) | | | |

Starting conditions: continuous culture of C. autoethanogenum,
containing 3.3 g/l acetate and 0.0 g/l ethanol at pH 5.5.

In the bioreactor bottles at 30 psi to 40 psi, about 2 g/l acetate and 0.6 g/l ethanol were produced and the pressure drop in the headspace was about 17 psi. This indicates that a substantial amount of the CO has been used for acetate production.

Surprisingly, at 50 psi, about 3 g/l acetate was consumed and 2.6 g/l ethanol produced. The results indicate that there is an optimum threshold CO partial pressure at which acetate to alcohol conversion occurs for an extended period. As CO concentration is proportional to CO partial pressure, the results indicate there is a sufficient CO concentration threshold at which C. auto converts acids to alcohols. However, it should be noted that lower pressure systems may also convert acids to alcohol, but as CO becomes depleted acetate production prevails. Additionally, under particularly CO (or H₂) depleted conditions, the culture may reconsume alcohol to produce acetate.

### Example 4B—Effect of CO Partial Pressure on Alcohol Production

Based on these results, a similar fermentation was conducted using the same gaseous substrate with media supplemented with different carbon sources. Serum vials were prepared in accordance with the above. Each serum vial was pressurized to 40 or 50 psia using a 95% CO in CO₂ gas mixture and incubated at 37° C. with constant shaking. A control bioreactor bottle (A) was unsupplemented, while other bottles were supplemented with some fructose (B), xylose (C) or pyruvate (D). These bottles were incubated at 37° C. with constant agitation. The metabolites and biomass concentrations, as well as the headspace overpressure and pH, were measured at the start of the fermentation and after 40 hours. Results at 40 psia are shown in Table 5 and results at 50 psia are shown in Table 6.

TABLE 5
Metabolism of 40 psia overpressure of a gaseous substrate comprising
95% CO in CO₂ by a culture of C. autoethanogenum in a serum
bottle at pH 5.5 after 40 h of fermentation.

| | | | | Over- | | |
|---|---|---|---|---|---|---|
| A | Acetate | Ethanol | Biomass | pressure | pH | Supplement |
| Start | 6.3 | 0.4 | 0.7 | 25 | 5.5 | — |
| End | 10.0 | 1.0 | 0.7 | 7 | 4.6 | — |
| Difference | +3.7 | +0.6 | +0.0 | −18 | −0.9 | — |
| | | | | Over- | | |
| B | Acetate | Ethanol | Biomass | pressure | pH | Fructose |
| Start | 6.3 | 0.5 | 0.7 | 25 | 5.5 | 0.9 |
| End | 10.1 | 1.7 | 0.7 | 9 | 4.6 | 0.0 |
| Difference | +3.8 | +1.2 | +0.0 | −16 | −0.9 | −0.9 |
| | | | | Over- | | |
| C | Acetate | Ethanol | Biomass | pressure | pH | Xylose |
| Start | 6.1 | 0.4 | 0.7 | 25 | 5.5 | 0.8 |
| End | 9.8 | 1.4 | 0.9 | 7 | 4.6 | 0.1 |
| Difference | +3.7 | +1.0 | +0.2 | −18 | −0.9 | −0.7 |
| | | | | Over- | | |
| D | Acetate | Ethanol | Biomass | pressure | pH | Pyruvate |
| Start | 7.0 | 0.0 | 0.8 | 25 | 5.5 | 0.8 |
| End | 10.1 | 0.8 | 0.7 | 8 | 4.8 | 0.0 |
| Difference | +3.1 | +0.8 | −0.1 | −17 | −0.7 | −0.8 |

Starting conditions: continuous culture of C. autoethanogenum at dilution rate = 0.04 h⁻¹, continuous flow of gaseous substrate comprising 95% CO in CO₂ (no overpressure), producing acetate and ethanol at pH 5.5. Data for acetate, ethanol, fructose, xylose, and pyruvate are concentrations in gram per litre. Biomass is given as gram of cell dry weight per litre. Overpressure of gas in the headspace is shown in psig.

In all the bioreactor bottles at 40 psia, for all conditions tested here, about 3.5 g/l acetate and minor amounts of ethanol were produced. The pressure drop in the headspace was about 17 psig. This indicates that a substantial portion of the CO has been consumed for acetate production. The pH decreased by about 0.9 units to 4.6. In all cases, there was minimal microbial growth.

TABLE 6
Metabolism of 50 psia overpressure of a gaseous substrate comprising
95% CO in CO₂ by a culture of C. autoethanogenum in a serum
bottle at pH 5.5 after 40 h of fermentation.

| | | | | Over- | | |
|---|---|---|---|---|---|---|
| A | Acetate | Ethanol | Biomass | pressure | pH | Supplement |
| Start | 6.3 | 0.4 | 0.7 | 35 | 5.5 | — |
| End | 1.1 | 4.3 | 0.4 | 25 | 6.4 | — |
| Difference | −5.2 | +3.9 | −0.3 | −10 | +0.9 | — |
| | | | | Over- | | |
| B | Acetate | Ethanol | Biomass | pressure | pH | Fructose |
| Start | 6.3 | 0.5 | 0.7 | 35 | 5.5 | 0.9 |
| End | 1.9 | 3.9 | 0.4 | 28 | 6.4 | 0.0 |
| Difference | −4.4 | +3.4 | −0.3 | −7 | +0.9 | −0.9 |
| | | | | Over- | | |
| C | Acetate | Ethanol | Biomass | pressure | pH | Xylose |
| Start | 6.1 | 0.4 | 0.7 | 35 | 5.5 | 0.8 |
| End | 2.8 | 3.4 | 0.4 | 30 | 6.4 | 0.0 |
| Difference | −3.3 | +3.0 | −0.3 | −5 | +0.9 | −0.8 |
| | | | | Over- | | |
| D | Acetate | Ethanol | Biomass | pressure | pH | Pyruvate |
| Start | 7.0 | 0.0 | 0.8 | 35 | 5.5 | 0.8 |
| End | 1.5 | 4.5 | 0.4 | 26 | 6.5 | 0.0 |
| Difference | −5.5 | +4.5 | −0.4 | −9 | +1.0 | −0.8 |

Starting conditions: continuous culture of C. autoethanogenum at dilution rate = 0.04 h⁻¹, continuous flow of gaseous substrate comprising 95% CO in CO₂ (no overpressure), producing acetate and ethanol at pH 5.5. Data for acetate, ethanol, fructose, xylose, and pyruvate are concentrations in gram per litre. Biomass is given as gram of cell dry weight per litre. Overpressure of gas in the headspace is shown in psig.

In all the bioreactor bottles at 50 psia, for all conditions tested here, significant amounts of acetate were consumed, and more than 3 g/l ethanol was produced. There is a strong correlation between acetate consumption and ethanol production. Acetate consumption/ethanol production occurs in such a way that for each mole of acetate consumed approximately one mole of ethanol was produced (Table 7). However, the supplemented carbohydrate (or pyruvate) was substantially consumed, and the biomass levels, estimated by optical density, decreased. In each instance, the pressure drop in the headspace was below 10 psi. In all cases, the pH increased by about 0.9 units to 6.4.

TABLE 7
Molar ratios for batch fermentation at 35 psi overpressure,
based on results shown in Table 2 and 3.

| | | Gas and | Gas and | Gas and |
|---|---|---|---|---|
| | Gas only | fructose | xylose | pyruvate |
| 1. Acetate | 0.83 | 0.70 | 0.54 | 0.79 |
| consumed/ | | | | |
| Acetate at start | | | | |
| 2. Ethanol | 0.81 | 0.70 | 0.64 | 0.84 |
| produced/ | | | | |
| Acetate at start | | | | |
| 3. Ethanol | 0.98 | 1.01 | 1.19 | 1.07 |
| produced/ | | | | |
| Acetate | | | | |
| consumed | | | | |

Given the acetate consumed/acetate at start (row 1), at least 50% and in some cases over 75% of the acetate present in the fermentation broth is consumed at elevated pressure. Given the ethanol produced/acetate at start (row 2), at least 60% and in some cases at least 80% of the consumed acid is replaced by alcohol. Given ethanol produced/acetate consumed (row 3), there is a strong correlation between the amount of acetate consumed in the fermentation process, and the alcohol produced. The theoretical level of dissolved CO in the media at 40 and 50 psia headspace overpressure was calculated in Table 8 based on the Henry's law.

TABLE 8
Calculation of the dissolved CO concentration
in media at different headspace overpressure
of a gaseous substrate comprising 95% CO in CO₂.

| | | | |
|---|---|---|---|
| Overpressure in | psia | 40 | 50 |
| headspace | | | |
| Partial pressure | psi | 37.7 | 47.2 |
| of CO in | | | |
| headspace | | | |
| Dissolved CO | mmol/l | 2.43 | 3.05 |
| concentration in | | | |
| media | | | |

Henry's constant for CO in water at 298 K is 1052.6 L · atm · mol⁻¹

The results presented here demonstrate that there is a CO partial pressure above which the metabolism of C. autoethanogenum changes substantially from production of acetate and biomass from the CO substrate to the conversion of at least a portion of acetate into ethanol. Thus, for a CO partial pressure below 37 psi, acetate and biomass are the major products of CO gas metabolism and pH becomes acidic, and further growth is inhibited. When the CO partial pressure is above 37 psi, biomass growth and acetate production appears to be inhibited, and consumption of acetate occurs. Furthermore, ethanol production occurs with minor CO consumption. At the same time, pH increases until it reaches 6.5, where the bacteria appear to be substantially inhibited and the conversion of acetate to ethanol stops. There is no noticeable effect of fructose, xylose or pyruvate at the concentration tested.

### Example 4C—Effect of CO Partial Pressure on Alcohol Production

Serum vials were prepared in accordance with the above. Each serum vial was pressurized to 25 psig (40 psia) with the indicated gas and incubated at 37° C. with constant shaking. Samples of the fermentation broth were taken at intervals of 1 h, 3 h and 5 h (see Table 9).

TABLE 9
Conversion of acetate to alcohol at various CO partial pressures
by Clostridium autoethanogenum over 5 hours.

| Gas | | Acetate | Ethanol | Pressure |
|---|---|---|---|---|
| composition | Time | conc (g/L) | conc (g/L) | (psig) |
| 100% CO | 0 | 11.914 | 0 | 25 |
| | 1 | 11.273 | 0.523 | 25.1 |
| | 3 | 10.488 | 1.295 | 22.7 |
| | 5 | 10.337 | 1.518 | 21.4 |
| 90% CO; 10% | 0 | 11.914 | 0 | 25 |
| N₂ | | | | |
| | 1 | 11.177 | 0.548 | 23.3 |
| | 3 | 10.407 | 1.315 | 21.2 |
| | 5 | 10.12 | 1.602 | 19.5 |
| 80% CO; 20% | 0 | 11.914 | 0 | 25 |
| N₂ | | | | |
| | 1 | 11.389 | 0.44 | 23.9 |
| | 3 | 11.042 | 1.055 | 22.5 |
| | 5 | 10.605 | 1.267 | 21.3 |
| 70% CO; 30% | 0 | 11.914 | 0 | 25 |
| N₂ | | | | |
| | 1 | 11.341 | 0.538 | 25.8 |
| | 3 | 10.51 | 1.193 | 23.4 |
| | 5 | 10.579 | 1.445 | 21.8 |
| 60% CO; 40% | 0 | 11.914 | 0 | 25 |
| N₂ | | | | |
| | 1 | 11.311 | 0.565 | 26.3 |
| | 3 | 10.959 | 1.297 | 23.5 |
| | 5 | 10.493 | 1.5 | 21.6 |
| 50% CO; 50% | 0 | 11.9 | 0 | 25 |
| N₂ | | | | |
| | 1 | 11.3 | 0.533 | 25.9 |
| | 3 | 11.0 | 1.236 | 23.5 |
| | 5 | 10.5 | 1.448 | 21.9 |

The results indicate that the sufficient threshold CO partial pressure at which acids are converted to alcohols is less than 20 psia. Over a short reaction time scale (c.f. Examples 4A-C), acetate is converted to alcohol substantially stoichiometrically at all CO partial pressures tested. Accordingly, a CO partial pressure over 20 psia is sufficient for C. auto to convert acids to alcohols.

### Example 5—Effect of Gas Composition

### Example 5A—Effect of Pure Gas on Acetate Conversion to Ethanol

Serum vials were prepared in accordance with the above. Each serum vial was pressurized to 25 psig (40 psia) with the indicated gas and incubated at 37° C. with constant shaking. Samples of the fermentation broth were taken at intervals of 1 h, 3 h and 5 h (see Table 10).

TABLE 10
Conversion of acetate to ethanol by Clostridium autoethanogenum
using alternative gas compositions.

| Gas | | Acetate | Ethanol | Pressure |
|---|---|---|---|---|
| composition | Time | conc (g/L) | conc (g/L) | (psig) |
| 100% N₂ | 0 | 12.531 | 0.133 | 26.1 |
| | 1 | 12.742 | 0 | 27.5 |
| | 3 | 12.394 | 0 | 27.1 |
| | 5 | 12.551 | 0 | 26.6 |
| 100% H₂ | 0 | 12.531 | 0.133 | 25.8 |
| | 1 | 11.921 | 0.384 | 24.8 |
| | 3 | 11.811 | 0.527 | 23.3 |
| | 5 | 11.998 | 0.546 | 22.5 |
| Steel Mill | 0 | 12.531 | 0.133 | 24.8 |
| Waste | | | | |
| Gas (approx. | 1 | 11.256 | 1.007 | 23.6 |
| 53% CO; | | | | |
| 18% CO₂; 26% | 3 | 10.668 | 1.605 | 20.6 |
| N₂; | | | | |
| 3%_{H2}) | 5 | 11.688 | 1.362 | 16.7 |

The results clearly indicate a reducing gas, such as CO or H₂ is necessary in order for C. auto to convert acids to alcohols. It is considered that hydrogen can be used in place of CO as the metabolic pathway from acids to alcohols includes hydrogenase enzymes. It is further considered that while H₂ is a suitable energy source for converting acids to alcohols it would not be adequate for biosynthesis and/or acetate production, which requires a carbon source as well as an energy source.

### Example 5B—Effect of Gas Composition on Ethanol Production

Serum vials were prepared in accordance with the above. However, prior to inoculation, the vials were spiked with butyric acid solution buffered to pH 5.5 with NaOH(aq). Initial concentrations at t=0 were acetate 6.7 g/l and butyrate 0.8 g/L (no ethanol or butanol was present). Samples of the fermentation broth were taken at 24 h (see Table 11).

TABLE 11
Conversion of acids to alcohols under different
CO partial pressures by Clostridium autoethanogenum,
in the presence and absence of hydrogen.

| Gas | Acetate | Ethanol | Butyrate | Butanol |
|---|---|---|---|---|
| composition | conc (g/L) | conc (g/L) | conc (g/L) | conc (g/L) |
| 100% CO | 4.7 | 1.8 | 0.4 | 0.3 |
| (40 psia) | | | | |
| 100% CO | 4.2 | 1.9 | 0.3 | 0.4 |
| (50 psia) | | | | |
| 75% CO 25% | 5.5 | 1.3 | 0.5 | 0.2 |
| H₂ (40 psia) | | | | |
| 60% CO 40% | 5.0 | 1.6 | 0.5 | 0.5 |
| H₂ (50 psia) | | | | |

Acids, such as butyric and acetic acids, can be converted to alcohols including ethanol and butanol in the presence of mixed CO/H₂ substrates. Clearly, in the absence of H₂, increased CO partial pressure improves overall conversion. However, the presence of H₂, particular at elevated partial pressure also improves overall conversion.

### Example 5C—Effect of Gas Composition on Ethanol Production

Serum vials were prepared in accordance with the above. Each serum vial was pressurized to 35 psig (50 psia) with the indicated gas and incubated at 37° C. with constant shaking. Samples of the fermentation broth were taken at 18 h (see Table 12).

TABLE 12
Conversion of acetate into ethanol under different
CO partial pressures by Clostridium autoethanogenum,
in the presence and absence of hydrogen.

| | Change in | | Change in | | End gas |
|---|---|---|---|---|---|
| Gas | acetate | | alcohol | | pressure (psia) |
| composition | conc (g/L) | conc (g/L) | CO | H₂ | CO₂ |
| 100% CO | −1.6 | +2.3 | 37 | — | 9 |
| 40% CO; | −1.2 | +2.5 | 0 | 8 | 11 |
| 40% H₂; | | | | | |
| 20% N₂ | | | | | |

Mixed substrates comprising CO and H₂ can be used to convert acids to alcohols in the presence of C. autoethanogenum. Interestingly, over the time scale of the experiment, significantly more alcohol is produced than acetate is consumed. This indicates that while acetate may be stoichiometrically converted into ethanol, additional acetate accumulates and may be converted to alcohol until CO is completely consumed.

### Example 5D—Effect of CO and H₂ Partial Pressure on Alcohol Production

Serum vials were prepared in accordance with the above. Each serum vial was pressurized to 35 psig (50 psia) with the indicated gas and incubated at 37° C. with constant shaking. Samples of the fermentation broth were taken at intervals of 1.5 h, 3 h, 5 h and 24 h (see Table 13).

TABLE 13
Conversion of acetate into ethanol under different CO and
H₂ partial pressures by Clostridium autoethanogenum

| Gas | | Acetate | Ethanol | Pressure |
|---|---|---|---|---|
| composition | Time | conc (g/L) | conc (g/L) | (psig) |
| 80% CO; 20% | 0 | 10.8 | 0.2 | 35 |
| H₂ | | | | |
| | 1.5 | 9.5 | 1.6 | 34.8 |
| | 3.25 | 9.3 | 2.4 | 32.2 |
| | 4.75 | 9.2 | 2.6 | 30.3 |
| | 6.75 | 9.4 | 2.5 | 28.7 |
| | 23 | 13.7 | 0.8 | 21.6 |
| 60% CO; 40% | 0 | 10.8 | 0.2 | 35 |
| H₂ | | | | |
| | 1.5 | 9.5 | 1.7 | 34.1 |
| | 3.25 | 9.5 | 2.6 | 30.6 |
| | 4.75 | 9.4 | 3.0 | 28.2 |
| | 6.75 | 9.5 | 3.1 | 25.4 |
| | 23 | 10.2 | 3.9 | 12.6 |

The results indicate that at elevated H₂ levels, there is an improvement in overall conversion of acids into alcohols. However, it is considered that ethanol is reconverted back to acetate as H₂ levels deplete over the course of the experiment, particularly at low levels of H₂ (e.g. 20%).

### Example 5E—Effect of CO Partial Pressure in Steel Mill Waste Gas

Serum vials were prepared in accordance with the above. Each serum vial was pressurized to 25 psig (40 psia) with steel mill waste gas (approx. 53% CO; 18% CO2; 26% N₂; 3% H₂) and incubated at 37° C. with constant shaking. Samples of the fermentation broth were taken at intervals of 1 h, 3 h and 5 h (see Table 14).

TABLE 14
Conversion of acetate to ethanol at different CO
partial pressures using steel mill waste gas.

| Initial Gas | | Acetate | Ethanol | Pressure | |
|---|---|---|---|---|---|
| Pressure | Time | conc (g/L) | conc (g/L) | (psig) | |
| 46 psia | 0 | 14.217 | 0.224 | 31 | 13.053 |
| | 1 | 13.335 | 0.696 | 31.2 | |
| | 3 | 12.775 | 1.811 | 26.4 | |
| | 5 | 13.053 | 2.197 | 21.2 | |
| 40 psia | 0 | 14.217 | 0.224 | 25 | |
| | 1 | 13.395 | 0.665 | 26.1 | |
| | 3 | 12.896 | 1.77 | 21.2 | |
| | 5 | 14.012 | 1.675 | 15.6 | |
| 30 psia | 0 | 14.217 | 0.224 | 15 | |
| | 1 | 13.485 | 0.623 | 15.9 | |
| | 3 | 12.909 | 1.742 | 11.9 | |
| | 5 | 14.363 | 1.364 | 8.2 | |

Steel mill waste gases can be used to convert acids into alcohols. Increasing CO partial pressure in the waste gas, has a beneficial effect on acid conversion.

### Example 5F—Effect of CO2 Partial Pressure on Ethanol Production

Serum vials were prepared in accordance with the above. Each serum vial was pressurized to 35 psig (50 psia) with the indicated gas and incubated at 37° C. with constant shaking. Samples of the fermentation broth were taken at intervals of 1 h, 3 h and 5 h (see Table 15).

TABLE 15
Conversion of acetate to ethanol at different CO₂ partial
pressures by Clostridium autoethanogenum.

| Gas | | Acetate | Ethanol | Pressure |
|---|---|---|---|---|
| composition | Time | conc (g/L) | conc (g/L) | (psig) |
| 40% CO; 60% N₂ | 0 | 9.256 | 0 | 35 |
| | 1 | 8.798 | 0.502 | 35.3 |
| | 3 | 8.31 | 1.076 | 33.3 |
| | 5 | 7.89 | 1.478 | 30.9 |
| 40% CO; 50% N₂; | 0 | 9.256 | 0 | 35 |
| 10% CO₂ | 1 | 8.721 | 0.509 | 35 |
| | 3 | 8.078 | 1.092 | 33.4 |
| | 5 | 7.69 | 1.511 | 31.1 |
| 40% CO; 40% N₂; | 0 | 9.256 | 0 | 35 |
| 20% CO₂ | 1 | 8.778 | 0.488 | 35.4 |
| | 3 | 8.115 | 1.057 | 32.9 |
| | 5 | 7.383 | 1.461 | 31.2 |
| 40% CO; 30% N₂; | 0 | 9.256 | 0 | 35 |
| 30% CO₂ | 1 | 8.763 | 0.473 | 34 |
| | 3 | 8.12 | 0.994 | 32.8 |
| | 5 | 7.769 | 1.4 | 31 |
| 40% CO; 20% N₂; | 0 | 9.256 | 0 | 35 |
| 40% CO₂ | 1 | 8.761 | 0.465 | 34 |
| | 3 | 8.191 | 0.962 | 32.9 |
| | 5 | 7.771 | 1.366 | 30.6 |
| | 0 | 9.256 | 0 | 35 |
| 40% CO; 10% N₂; | 1 | 9.255 | 0 | 34.5 |
| 50% CO₂ | 3 | 9.527 | 0.106 | 34 |
| | 5 | 9.131 | 0.235 | 33 |
| 40% CO; 60% CO₂ | 0 | 9.256 | 0 | 35 |
| | 1 | 8.814 | 0.384 | 32.2 |
| | 3 | 8.23 | 0.737 | 31.5 |
| | 5 | 8.365 | 1.046 | 30 |

Substrates comprising CO containing a variety of constituents can be used to convert acids into alcohols. However, it is noted that increased levels of CO₂ have a slight inhibitory effect on alcohol production.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology.

All patents, patent applications, and published references cited herein are hereby incorporated by reference in their entirety. The claims are intended to cover the components and steps in any sequence which is effective to meet the objectives there intended unless the context specifically indicates the contrary.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. All pressures disclosed herein are absolute unless otherwise stated. All temperatures are Celsius unless otherwise stated.

### EMBODIMENTS OF THE DISCLOSURE

Embodiment 1. A method of integrating a gas fermentation process with a chemical or refining process, the method comprising:
a. providing a gaseous carbon source from the chemical or refining process to the gas fermentation process; or
b. providing a gas fermentation product from the gas fermentation process to the chemical or refining process; or
c. both providing the gaseous carbon source from the chemical or refining process to the gas fermentation process and providing the gas fermentation product from the gas fermentation process to the chemical or refining process;
d. wherein the gas fermentation process utilizes a C1-fixing microorganism to ferment at least the gaseous carbon source from the chemical or refining process to produce the gas fermentation product; and
e. wherein the chemical or refining process generates a chemical or refining product.

Embodiment 2. The method of embodiment 1, wherein the gaseous carbon source is provided from the chemical or refining process to the gas fermentation process, the method further comprising adding the gas fermentation product to the chemical or refining process.

Embodiment 3. The method of embodiments 1 or 2, wherein the gas fermentation product is the same as the chemical or refining product.

Embodiment 4. The method of any of the preceding embodiments, comprising performing the gas fermentation process in a gas fermentation unit that receives the gaseous carbon source.

Embodiment 5. The method of any of the preceding embodiments, wherein the gas fermentation product comprises producing, by the gas fermentation process, a mixture comprising at least one of the gas fermentation product or a product-enriched off-gas.

Embodiment 6. The method of any of the preceding embodiments, wherein providing the gas fermentation product from the gas fermentation process to the chemical or refining process comprises recovering a carbon product from the mixture and adding the carbon product to the chemical or refining product.

Embodiment 7. The method of any of the preceding embodiments, wherein the C1-fixing microorganism is an anaerobic bacterium.

Embodiment 8. The method of any of the preceding embodiments, wherein the C1-fixing microorganism is an aerobic bacterium.

Embodiment 9. The method of any of the preceding embodiments, further comprising the step of determining the mass of the carbon product.

Embodiment 10. The method of any of the preceding embodiments, further comprising the step of determining the volume of the carbon product.

Embodiment 11. A gas fermentation system for integration with a chemical or refining process, comprising:
a first pipe configured to receive a gaseous carbon stream from a facility of the chemical or refining process;
a gas fermentation unit coupled with the first pipe to receive the gaseous carbon stream, the gas fermentation unit comprising a culture of at least one C1-fixing microorganism capable of producing an output comprising a gas fermentation product from the gaseous carbon stream; and
a second pipe coupled with the gas fermentation unit having a connection to a chemical plant and configured to contain the gas fermentation product;
wherein the second pipe is adapted to receive the output from the gas fermentation unit and transport the output to the chemical plant, such that the gas fermentation product can be isolated, extracted, processed, or any combination thereof at the chemical plant.

Embodiment 12. The system of embodiment 11, wherein the oil refinery or production facility and the chemical plant are geographically adjacent to each other.

Embodiment 13. The system of embodiments 11 or 12, wherein the oil refinery or production facility and the chemical plant are part of the same operating complex.

Embodiment 14. The system of any one of embodiments 11 to 13, wherein the oil refinery or production facility and the chemical plant are within 1 mile, within 2 miles, within 3 miles, within 4 miles, or within 5 miles of each other.

Embodiment 15. The system of any one of embodiments 11 to 14, wherein the gaseous carbon stream is a waste gas, flare gas, natural gas, or captured flared gas.

Embodiment 16. The system of any one of embodiments 11 to 15, wherein the gaseous carbon stream comprises CO, CO₂, methane, or any combination thereof, and, optionally, H₂.

Embodiment 17. The system of any one of embodiments 11 to 16, wherein the oil refinery or production facility is selected from a topping refinery, a hydro-skimming refinery, a conversion refinery, a deep conversion refinery, or a coking unit.

Embodiment 18. The system of any one of embodiments 11 to 17, wherein the gaseous carbon stream originates from a flexicoker, a pressure swing absorption unit, a flare stream, captured flared gases, or a pressure relief safety valve.

Embodiment 19. The system of any one of embodiments 11 to 18, wherein the C1-fixing microorganism is aerobic or anaerobic.

Embodiment 20. The system of any one of embodiments 11 to 19, wherein the C1-fixing microorganism is selected from a genus of Clostridium, Moorella, Carboxydothermus, Ruminococcus, Acetobacterium, Eubacterium, Butyribacterium, Oxobacter, Methanosarcina, Methanosarcina, Desulfotomaculum, and Cupriavidus.

Embodiment 21. The system of any one of embodiments 11 to 20, the chemical plant is selected from an olefins plant, a poly-olefins plant, a polypropylene plant, a polyethylene plant, a polymer plant, a high-density polyethylene plant, an oligomers plant, a nitriles plant, an oxides plant, or a styrenics plant.

Embodiment 22. The system of any one of embodiments 11 to 21, wherein the gas fermentation product is transported to a steam cracker within the chemical plant.

Embodiment 23. The system of any one of embodiments 11 to 22, wherein the gas fermentation product is selected from an alcohol, an acid, a diacid, an alkene, a terpene, an isoprene, and alkyne.

Embodiment 24. The system of any one of embodiments 11 to 23, wherein the gas fermentation product is selected from ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1propanol, 1hexanol, 1octanol, chorismate-derived products, 3hydroxybutyrate, 1,3butanediol, 2-hydroxyisobutyrate or 2-hydroxyisobutyric acid, isobutylene, adipic acid, 1,3hexanediol, 3-methyl-2-butanol, 2-buten-1-ol, isovalerate, isoamyl alcohol, and monoethylene glycol, or any combination thereof.

Embodiment 25. The system of any one of embodiments 11 to 24, further comprising a flow meter configured to determine a property or concentration of the gas fermentation product.

Embodiment 26. A method of integrating a gas fermentation unit with a chemical or refining processing unit, the method comprising:
providing a gaseous carbon source from a section of the chemical or refining processing unit;
fermenting a portion of the gaseous carbon source into a gas fermentation product with a gas fermentation unit, wherein the gas fermentation unit utilizes a C1-fixing microorganism to convert at least a portion of the gaseous carbon source into the gas fermentation product, resulting in a fermented mixture;
extracting the gas fermentation product from the fermented mixture; and adding the gas fermentation product to a product of the chemical or refining processing unit.

Embodiment 27. The method of embodiment 26, wherein the gas fermentation product is the same as the product of the chemical or refining process.

Embodiment 28. The method of embodiment 26 or 27, wherein producing, by the gas fermentation process, the gas fermentation product comprises producing, by the gas fermentation process, a mixture comprising at least one of the gas fermentation product or a product-enriched off-gas.

Embodiment 29. The method of any one of embodiments 26 to 28, wherein providing the gas fermentation product from the gas fermentation process to the chemical or refining process comprises extracting a carbon product from the mixture and adding the carbon product to the chemical or refining product.

Embodiment 30. The method of any one of embodiments 26 to 29, wherein the C1-fixing microorganism is an anaerobic bacterium.

Embodiment 31. The method of any one of embodiments 26- to, wherein the C1-fixing microorganism is an aerobic bacterium.

Embodiment 32. The method of any one of embodiments 26 to 31, further comprising the step of determining the mass of the gas fermentation product.

Embodiment 33. The method of any one of embodiments 26 to 32, further comprising the step of determining the volume of the gas fermentation product.

Embodiment 34. A gas fermentation system for integration with a chemical or refining process, comprising:
a first connection configured to receive a carbon source from the chemical or refining process;
a gas fermentation unit configured to receive a gaseous carbon stream of the carbon source and utilize a C1-fixing microorganism to convert the gaseous carbon stream into a gas fermentation product; and
a second connection configured to add the gas fermentation product to a product of the chemical or refining process.

Embodiment 35. The gas fermentation system of embodiment 34, wherein the carbon source comprises at least one of a solid or a liquid, and the gas fermentation system comprises a gasifier configured to generate the gaseous carbon stream from the carbon source.

Embodiment 36. The gas fermentation system of embodiments 34 or 35, wherein the gas fermentation product is the same as the product of the chemical or refining process.

Embodiment 37. The gas fermentation system of any one of embodiments 34 to 36, wherein the gas fermentation unit is configured to generate the gas fermentation product to comprise a product-enriched off-gas.

Embodiment 38. The gas fermentation system of any one of embodiments 34 to 37, wherein the gas fermentation unit is configured to generate a mixture comprising the gas fermentation product, the gas fermentation system further comprising an extractor configured to extract the gas fermentation product from the mixture to add the gas fermentation product to the product of the chemical or refining process.

Embodiment 39. The gas fermentation system of any one of embodiments 34 to 38, wherein the C1-fixing microorganism is an anaerobic bacterium.

Embodiment 40. The gas fermentation system of any one of embodiments 34 to 38, wherein the C1-fixing microorganism is an aerobic bacterium.

Embodiment 41. The gas fermentation system of any one of embodiments 34 to 40, further comprising a sensor configured to determine a mass of the gas fermentation product.

Embodiment 42. The gas fermentation system of any one of embodiments 34 to 41, further comprising a sensor configured to determine a volume of the gas fermentation product.

1. A method of integrating a gas fermentation process with a chemical or refining process, the method comprising:
a. providing a gaseous carbon source from the chemical or refining process to the gas fermentation process; or
b. providing a gas fermentation product from the gas fermentation process to the chemical or refining process; or
c. both providing the gaseous carbon source from the chemical or refining process to the gas fermentation process and providing the gas fermentation product from the gas fermentation process to the chemical or refining process;
d. wherein the gas fermentation process utilizes a C1-fixing microorganism to ferment at least the gaseous carbon source from the chemical or refining process to produce the gas fermentation product; and
e. wherein the chemical or refining process generates a chemical or refining product.

2. The method of claim 1, wherein the gaseous carbon source is provided from the chemical or refining process to the gas fermentation process, the method further comprising adding the gas fermentation product to the chemical or refining process.

3. The method of claim 1, wherein the gas fermentation product is the same as the chemical or refining product.

4. The method of claim 1, wherein the gas fermentation product comprises producing, by the gas fermentation process, a mixture comprising at least one of the gas fermentation product or a product-enriched off-gas.

5. The method of claim 4, wherein providing the gas fermentation product from the gas fermentation process to the chemical or refining process comprises recovering a carbon product from the mixture and adding the carbon product to the chemical or refining product.

6. The method of claim 1, wherein the C1-fixing microorganism is an anaerobic bacterium or an aerobic bacterium.

7. The method of claim 1, further comprising the step of determining the mass or the volume of the carbon product.

8. A gas fermentation system for integration with a chemical or refining process, comprising:
a first conduit configured to receive a gaseous carbon stream from a facility of the chemical or refining process;
a gas fermentation unit coupled with the first conduit to receive the gaseous carbon stream, the gas fermentation unit comprising a culture of at least one C1-fixing microorganism capable of producing an output comprising a gas fermentation product from the gaseous carbon stream; and
a second conduit coupled with the gas fermentation unit having a connection to a chemical plant and configured to contain the gas fermentation product;
wherein the second conduit is adapted to receive the output from the gas fermentation unit and transport the output to the chemical plant, such that the gas fermentation product can be isolated, separated, processed, or any combination thereof at the chemical plant.

9. The system of claim 8, wherein the oil refinery or production facility and the chemical plant are geographically adjacent to each other, part of the same operating complex, or both.

10. The system of claim 8, wherein the oil refinery or production facility and the chemical plant are within 1 mile, within 2 miles, within 3 miles, within 4 miles, or within 5 miles of each other.

11. The system of claim 8, wherein the gaseous carbon stream is a waste gas, flare gas, natural gas, or captured flared gas.

12. The system of claim 8, wherein the gaseous carbon stream comprises CO, CO₂, methane, or any combination thereof, and, optionally, H₂.

13. The system of claim 8, wherein the oil refinery or production facility is selected from a topping refinery, a hydro-skimming refinery, a conversion refinery, a deep conversion refinery, or a coking unit.

14. The system of claim 8, wherein the gaseous carbon stream originates from a flexicoker, a pressure swing absorption unit, a flare stream, captured flared gases, or a pressure relief safety valve.

15. The system of claim 8, wherein the chemical plant is selected from an olefins plant, a poly-olefins plant, a polypropylene plant, a polyethylene plant, a polymer plant, a high-density polyethylene plant, an oligomers plant, a nitriles plant, an oxides plant, or a styrenics plant.

16. The system of claim 8, wherein the gas fermentation product is selected from an alcohol, an acid, a diacid, an alkene, a terpene, an isoprene, and alkyne.

17. The system of claim 8, wherein the gas fermentation product is selected from ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1propanol, 1hexanol, 1octanol, chorismate-derived products, 3hydroxybutyrate, 1,3butanediol, 2-hydroxyisobutyrate or 2-hydroxyisobutyric acid, isobutylene, adipic acid, 1,3hexanediol, 3-methyl-2-butanol, 2-buten-1-ol, isovalerate, isoamyl alcohol, and monoethylene glycol, or any combination thereof.

18. The system of claim 8, further comprising a flow meter configured to determine a property or concentration of the gas fermentation product.

19. A method of integrating a gas fermentation unit with a chemical or refining processing unit, the method comprising:
providing a gaseous carbon source from a section of the chemical or refining processing unit;
fermenting a portion of the gaseous carbon source into a gas fermentation product with a gas fermentation unit, wherein the gas fermentation unit utilizes a C1-fixing microorganism to convert at least a portion of the gaseous carbon source into the gas fermentation product, resulting in a fermented mixture;
separating the gas fermentation product from the fermented mixture; and
adding the gas fermentation product to a product of the chemical or refining processing unit.

20. The method of claim 19, wherein the gas fermentation product is the same as the product of the chemical or refining process.

21. The method of claim 19 wherein the gas fermentation product comprises producing, by the gas fermentation process, a mixture comprising at least one of the gas fermentation product or a product-enriched off-gas.

22. The method of claim 19, wherein providing the gas fermentation product from the gas fermentation process to the chemical or refining process comprises separating a carbon product from the mixture and adding the carbon product to the chemical or refining product.

23. The method of claim 19, wherein the C1-fixing microorganism is an anaerobic or aerobic bacterium.

24. The method of claim 19, further comprising the step of determining the mass or volume of the gas fermentation product.

25. A gas fermentation system for integration with a chemical or refining process, comprising:
a first connection configured to receive a carbon source from the chemical or refining process;
a gas fermentation unit configured to receive a gaseous carbon stream of the carbon source and utilize a C1-fixing microorganism to convert the gaseous carbon stream into a gas fermentation product; and
a second connection configured to add the gas fermentation product to a product of the chemical or refining process.

26. The gas fermentation system of claim 25, wherein the carbon source comprises at least one of a solid or a liquid, and the gas fermentation system comprises a gasifier configured to generate the gaseous carbon stream from the carbon source.

27. The gas fermentation system of claim 25 wherein the gas fermentation unit is configured to generate a mixture comprising the gas fermentation product, the gas fermentation system further comprising a separator configured to separate the gas fermentation product from the mixture to add the gas fermentation product to the product of the chemical or refining process.

28. The gas fermentation system of claim 25, wherein the C1-fixing microorganism is an anaerobic or aerobic bacterium.

29. The gas fermentation system of claim 25, further comprising a sensor configured to determine a mass or a volume of the gas fermentation product.
